(19) United States
(12) Patent Application Publication
(10) Pub. No.: US 2025/0367430 A1
(43) Pub. Date: Dec. 4, 2025

(54) PUMP-INDEPENDENT PHYSIOLOGICAL CONTROLLER

(71) Applicants: Yale University, New Haven, CT (US); University of Louisville Research Foundation, Inc., Louisville, KY (US)

(72) Inventors: Pramod Bonde, New Haven, CT (US); Guruprasad Giridharan, Louisville, KY (US)

(21) Appl. No.: 19/219,019

(22) Filed: May 27, 2025

Related U.S. Application Data
(60) Provisional application No. 63/652,451, filed on May 28, 2024.

(51) Int. Cl. A61M 60/515; A61M 60/174; A61M 60/221; A61M 60/232; A61M 60/411; A61M 60/538; A61M 60/546

(52) U.S. Cl. CPC A61M 60/515; A61M 60/174; A61M 60/221; A61M 60/232; A61M 60/411; A61M 60/538; A61M 60/546

(57) ABSTRACT

A physiological control system for a blood pump includes a controller configured to receive an input signal indicative of ventricular chamber volume, and generate an output pump control signal based on the input signal. A physiological method for controlling a blood pump is also disclosed.

### CROSS-REFERENCE TO RELATED APPLICATIONS

This application claims priority to U.S. Provisional Application No. 63/652,451 filed on May 28, 2024, the contents of which are incorporated by reference herein in its entirety.

### BACKGROUND OF THE INVENTION

LVAD therapy using small rotary blood pumps (RBP) has radically improved congestive heart failure survival with improved reliability and lower morbidity over earlier larger pulsatile pumps. For the last two decades, LVAD has been a vital treatment option, with more than 4,000 implants per year in the US with >60% five-year survival. Rotary blood pumps currently operate at a fixed pump speed. Thus, RBP are unable to meet physiologic demand and susceptible to ventricular suction. The control of LVAD has always been in a fixed pump speed mode, although heart failure is a dynamic process. Once the pump is implanted, patients are sent back home with a pre-set pump speed. Exercise capacity and peak oxygen consumption following LVAD implantation remains considerably restricted and fixed speed operation results in frequent suction events. An insufficient rise in cardiac output in response to increased flow demand is one of the most important factors that has potential for modification.

The controllers of current generation LVADs do not change the rotation speed based on hemodynamic parameters, volume shifts or heart rate (HR), and may not cover exertional needs. Furthermore, the diminished vascular pulsatility due to rotary LVAD support has been associated with adverse events including endothelial dysfunction and gastrointestinal bleeding. The typical method of power transfer either via wired or wireless connection has been supplying a fixed amount, which has never been changed since the invention of electricity. However, the load uses only a part of it and the rest is normally wasted as heat. Inability to sense the load and control the pump in response to the severity of the congestive heart failure has been Achilles' heel of LVAD technology. Current sensors based on piezoelectric, ultrasound, or infra-red are bulky and impractical to implant, while motor current sensing is inconsistent.

RBP are currently used as bridge to transplantation and destination therapy for end-stage heart failure due to the scarcity of donor hearts. RBP operate at a user-defined constant pump speed setpoint clinically, resulting in poor adaptation of RBP generated flow rates to meeting cardiac demand. RBP have low sensitivity preload and afterload sensitivity compared to the native heart [10,11]. Thus, during moderate physical activity, RBP can generated flow rates may be inadequate to meet cardiac demand, resulting in hypoperfusion, LV volume overload, and higher ventricular wall stress [12]. RBP flows in excess of venous return can lead to suction events, which can result in arrhythmias, ventricular damage, and death. Thus, RBP support needs to adapt to constantly changing physiologic needs based on activity levels, circadian rhythm, etc. while avoiding ventricular suction [13].

RBPs are the mechanical circulation support devices implanted in heart failure (HF) patients between the sick left ventricle (LV) and the aorta for increasing cardiac output (CO) and reducing ventricular workload. For HF patients, RBPs have been accepted widely as the transition of heart transplantation, cardiac recovery, or a long-term implantation (destination therapy) because of their simpler design and smaller size that can decrease surgical trauma, reduce power consumption and thrombosis rate, and improve durability and reliability [6-9]. However, the problem for insufficient physiologic perfusion and LV suction (ventricular collapse) events may be the significant clinical issues after implanting the RBP due to its markedly low sensitivity of preload and afterload compared to the native heart [10,11]. Insufficient pump flow rate may result in hypoperfusion of end-organs, LV volume overload, tissue hypoxia and injury, organ failure, and cell death [12], while the occurrence of LV suction may lead to some serious adverse events that can happen in the cardiovascular system and within the RBPs [13].

There have been different algorithms of physiological control and suction prevention for the RBPs in various aspects, for example, based on pump flow (PF) [14,15], pump speed (PS) [16,17], and pressure signals [18,19], with different methods such as machine learning [20-22], fuzzy logic controller [23,24], PI/PID [25,26], and achieved using in-silico [14-26], in-vitro [27-31], and in-vivo models [32-34]. In addition, some of the control algorithms are sensorless-based [20,24,32,35], and some can also enhance the vascular pulsatility as the constant speed of RBPs caused [19,36-38]. All of these control strategies can be used for left/right ventricle, or both [39-41], and Fontan failure syndrome [42,43]. While these control algorithms may have some common disadvantages and shortcomings, the more crucial problem is that these control algorithms have not demonstrated their pump-independent performance, i.e., these control algorithms may be suitable for only one type of RBPs (axial flow or centrifugal flow pump). There have been pump-independent control algorithms, however, some of them were sensor-based [39,44], which usually need flow or pressure sensors, but they cannot be implanted for a long time due to sensor drift or failure, repeated calibrations, thrombus formation, and induction of septicemia. In addition, sensors implantation increased the system complexity and decreased the overall reliability [45,46]. For other control algorithms using two types of RBPs [47,48], one of them is a mixed-flow pump, not as regularly used as an axial or centrifugal RBP.

Accordingly, there is a need in the art for an improved system, device and method for addressing these conventional limitations for controlling LVAD devices. Embodiments described herein fit this need.

### SUMMARY OF THE INVENTION

In one embodiment, a physiological control system for a blood pump includes a controller configured to receive an input signal indicative of ventricular chamber volume, and generate an output pump control signal based on the input signal. In one embodiment, the measured ventricular chamber volume is at least one of end diastolic volume, end systolic volume, mean ventricular volumes, stroke volume, or ventricular volume. In one embodiment, the output pump control signal is generated based on the gain-scheduling proportional-integral control equation:

$$I = {K}_{P} ( EDV - {EDV}_{r} ) + {K}_{I} {\int }_{0}^{t} ( EDV - {EDV}_{r} ) dt .$$

In one embodiment, EDVr is substantially 85 ml. In one embodiment, the output pump control signal is based on weighting ventricular chamber volumes dependent of the part of the cardiac cycle. In one embodiment, KP is substantially 0.01 and KI is substantially 0.002 for controlling an axial rotary blood pump. In one embodiment, KP is substantially 0.03 and KI is substantially 0.006 for controlling a centrifugal rotary blood pump. In one embodiment, the controller calculates setpoints as at least one of a constant setpoint, a repeating continuous or discrete function, or a non-repeating function. In one embodiment, the measured chamber volume is based on a signal generated from resonantly coupled sensors. In one embodiment, the resonantly coupled sensors comprise apical and outflow sensors. In one embodiment, the output pump control signal is a pump speed signal. In one embodiment, the controller is configured to detect when end-systolic volumes are above a minimum setpoint. In one embodiment, the controller is configured to detect changes in pump power. In one embodiment, the controller is configured to periodically switch volume setpoints to generate pulsatility. In one embodiment, the controller is configured to periodically set pump speed at a low constant speed for estimating at least one of the ejection fraction, rate of change of volume, ventricular end-systolic and end-diastolic volumes. In one embodiment, the controller is configured to use stroke volume as a setpoint that is periodically increased to a larger value. In one embodiment, the controller is configured to set pump flow lower than stroke volume. In one embodiment, the controller is configured to increase stroke volume as improvement in physiological parameters is detected.

In one embodiment, a physiological method for controlling a blood pump includes the steps of receiving an input signal indicative of ventricular chamber volume; and generating an output pump control signal based on the input signal. In one embodiment, the measured ventricular chamber volume is at least one of end diastolic volume, end systolic volume, mean ventricular volumes, stroke volume, or ventricular volume. In one embodiment, the output pump control signal is generated based on the gain-scheduling proportional-integral control equation

$$I = {K}_{P} ( EDV - {EDV}_{r} ) + {K}_{I} {\int }_{0}^{t} ( EDV - {EDV}_{r} ) dt .$$

In one embodiment, the output pump control signal is based on weighting ventricular chamber volumes dependent of the part of the cardiac cycle. In one embodiment, EDVr is substantially 85 ml. In one embodiment, KP is substantially 0.01 and KI is substantially 0.002 for controlling an axial rotary blood pump. In one embodiment, KP is substantially 0.03 and KI is substantially 0.006 for controlling a centrifugal rotary blood pump. In one embodiment, the controller calculates setpoints as at least one of a constant setpoint, a repeating continuous or discrete function, or a non-repeating function. In one embodiment, the measured chamber volume is based on a signal generated from resonantly coupled sensors. In one embodiment, the resonantly coupled sensors comprise apical and outflow sensors. In one embodiment, the output pump control signal is a pump speed signal. In one embodiment, the method includes the step of detecting when end-systolic volumes are above a minimum setpoint. In one embodiment, the method includes the step of detecting changes in pump power. In one embodiment, the method includes the step of periodically switching volume setpoints to generate pulsatility. In one embodiment, the method includes the step of periodically setting pump speed at a low constant speed for estimating at least one of the ejection fraction, rate of change of volume, ventricular end-systolic and end-diastolic volumes. In one embodiment, the method includes the step of utilizing stroke volume as a setpoint that is periodically increased to a larger value. In one embodiment, the method includes the step of setting pump flow lower than stroke volume. In one embodiment, the method includes the step of increasing stroke volume as improvement in physiological parameters is detected.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1A is a schematic diagram of the LPM of cardiovascular system couple with the RBP control system according to one embodiment.

FIG. 1B is a volume (V) based differential equation (1) where in module n the dVₙ/dt represents the changing rate of V, and Fⁱⁿ and F^{out} denote blood flow into and out of module n, respectively according to one embodiment.

FIGS. 1C and 1D show differential equations (2) and (3) regarding the rotational speed and flow of the pump respectively for the axial RBP model according to one embodiment.

FIGS. 1E and 1F show differential equations (4) and (5) regarding the rotational speed and flow of the pump respectively for the centrifugal RBP model according to one embodiment.

FIG. 1G shows a control law equation (6) to implement the EDV control algorithm according to one embodiment.

FIG. 1H shows an apical cuff with integrated apical and outflow sensors according to one embodiment.

FIG. 2A shows graphical in-silico results of the pump-independent control strategy at rest for (a)-(c) the axial RBP without LVV measurement noise, (d)-(f) the axial RBP with LVV measurement noise, (g)-(i) the centrifugal RBP without LVV measurement noise, (j)-(I) the centrifugal RBP with LVV measurement noise according to one embodiment.

FIG. 2B (Table 1) shows results of the pump-independent EDV control algorithm under varying conditions for the axial RBP, and FIG. 2C (Table 2) shows results of the pump-independent EDV control algorithm under varying conditions for the centrifugal RBP.

FIG. 3 shows in-silico results of the pump-independent control algorithm at rest under the condition of 8-time PVR starting at t=150 s for (a)-(c) the axial RBP without LVV measurement noise, (d)-(f) the axial RBP with LVV measurement noise, (g)-(i) the centrifugal RBP without LVV measurement noise, (j)-(l) the centrifugal RBP with LVV measurement noise.

FIG. 4 shows in-silico results of the pump-independent control algorithm during the transient change from exercise to rest starting at t=150 s for (a)-(c) the axial RBP without LVV measurement noise, (d)-(f) the axial RBP with LVV measurement noise, (g)-(i) the centrifugal RBP without LVV measurement noise, (j)-(l) the centrifugal RBP with LVV measurement noise.

FIG. 5 shows in-silico results of the proposed pump-independent control algorithm for 80 ml setpoint during the transient change from exercise to rest starting at t=150 s for (a)-(c) the axial RBP without LVV measurement noise, (d)-(f) the axial RBP with LVV measurement noise, (g)-(i) the centrifugal RBP without LVV measurement noise, (j)-(l) the centrifugal RBP with LVV measurement noise, according to one embodiment.

FIG. 6 (Table 3) shows the comparison between the EDV control algorithm and other previous control strategies, according to one embodiment.

FIGS. 7A-7L depict in-silico results of the pump-independent control strategy without pulsatility at rest, according to one embodiment. FIGS. 7A-7C depict in-silico results for the axial RBP without LVV measurement noise. FIGS. 7D-7F depict in-silico results for the axial RBP with LVV measurement noise. FIGS. 7G-7I depict in-silico results for the centrifugal RBP without LVV measurement noise. FIGS. 7J-7L depict in-silico results for the centrifugal RBP with LVV measurement noise.

FIGS. 8A-8L illustrate the performance of the pump-independent control algorithm operating in pulsatility mode for both axial and centrifugal LVADs at rest, with and without LVV measurement noise, according to one embodiment. FIGS. 8A-8L depict in-silico results of the pump-independent control strategy with pulsatility at rest. FIGS. 8A-8C depict in-silico results for the axial flow LVAD without LVV measurement noise. FIGS. 8D-8F depict in-silico results for the axial flow LVAD with LVV measurement noise. FIGS. 8G-8I depict in-silico results for the centrifugal flow LVAD without LVV measurement noise. FIGS. 8J-8L depict in-silico results for the centrifugal flow LVAD with LVV measurement noise.

FIG. 9 (Table 4) shows results of the pump-independent EDV control algorithm for the axial flow LVAD under various physiological conditions, with and without 6% LVV measurement noise, according to one embodiment.

FIG. 10 (Table 5) shows results of the pump-independent EDV control algorithm for the centrifugal flow LVAD under various physiological conditions, with and without 6% LVV measurement noise, according to one embodiment.

FIG. 11 (Table 6) shows the effect of ESV_{Hr} modulation (90 vs. 80 mL) on pulsatility characteristics in axial and centrifugal LVADs under varying noise conditions, according to one embodiment.

FIGS. 12A-12L show in-silico results of the pump-independent control algorithm at rest under the condition of 8-time PVR starting at t=150 s for the axial LVAD, according to one embodiment. FIGS. 12A-12C show in-silico results without pulsatility and without LVV measurement noise. FIGS. 12D-12F show in-silico results without pulsatility and with LVV measurement noise. FIGS. 12G-12I show in-silico results with pulsatility and without LVV measurement noise. FIGS. 12J-12L show in-silico results with pulsatility and with LVV measurement noise.

FIGS. 13A-13L show in-silico results of the pump-independent control algorithm at rest under the condition of 8-time PVR starting at t=150 s for the centrifugal LVAD, according to one embodiment. FIGS. 13A-13C show in-silico results without pulsatility and without LVV measurement noise. FIGS. 13D-13F show in-silico results without pulsatility and with LVV measurement noise. FIGS. 13G-13I show in-silico results with pulsatility and without LVV measurement noise. FIGS. 13J-13L show in-silico results with pulsatility and with LVV measurement noise.

FIGS. 14A-14L show in-silico results of the pump-independent control algorithm during the transient change from exercise to rest at t=150 s for the axial LVAD, according to one embodiment. FIGS. 14A-14C show in-silico results without pulsatility and without LVV measurement noise. FIGS. 14D-14F show in-silico results without pulsatility and with LVV measurement noise. FIGS. 14G-14I show in-silico results with pulsatility and without LVV measurement noise. FIGS. 14J-14L show in-silico results with pulsatility and with LVV measurement noise. FIGS. 15A-15L show in-silico results of the pump-independent control algorithm during the transient change from exercise to rest at t=150 s for the centrifugal LVAD.

FIGS. 15A-15C show in-silico results without pulsatility and without LVV measurement noise, according to one embodiment. FIGS. 15D-15F show in-silico results without pulsatility and with LVV measurement noise. FIGS. 15G-15I show in-silico results with pulsatility and without LVV measurement noise. FIGS. 15J-15L show in-silico results with pulsatility and with LVV measurement noise. Table

### DETAILED DESCRIPTION OF THE INVENTION

It is to be understood that the figures and descriptions of the present invention have been simplified to illustrate elements that are relevant for a more clear comprehension of the present invention, while eliminating, for the purpose of clarity, many other elements found in systems and methods of physiological controllers. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein is a physiological controller device and method.

Embodiments of the physiological controller described herein utilize several components that work synergistically, including novel resonantly coupled sensors (within the apical cuff, septum, and outflow graft sleeve) with high fidelity and minimal long-term drift for the remote monitoring of cardiac chamber volume to achieve autonomous powering and to enable control of LVAD, and an algorithm that operates in response to physiological needs based on ventricular chamber size and simultaneously avoids ventricular suction.

Embodiments of the physiological controller described herein include a left ventricular end-diastolic volume (LVEDV) based physiologic control algorithm using resonantly coupled high-efficiency sensors. The resonantly coupled sensors consists of apical and outflow sensors that can accurately assess the ventricular chamber size with minimal long-term drift (˜1%) at 9 months. The ability of the LVEDV based control algorithm was evaluated using an in-silico model of the human circulatory system coupled to an axial or centrifugal flow pump with 15% uniformly distributed volume measurement noise. The LVEDV setpoint was set to 85 ml, and the efficacy of the LVEDV control algorithm was evaluated and compared to constant pump speed control strategy during (1) rest and exercise states; (2) rapid, eight-fold augmentation of pulmonary vascular resistance; and (3) rapid change in physiologic states between rest and exercise. Safety and robustness of the algorithm was also evaluated by assuming a 6% volume drift (80 ml LVEDV setpoint). The LVEDV control algorithm provided sufficient physiological perfusion simultaneously and avoided ventricular suction in all cases. Performance of the LVEDV algorithm was superior compared to maintaining constant pump speed for both types of LVAD, demonstrating pump independence of the algorithm.

The physiological control system in one embodiment includes a controller configured to receive an input signal indicative of ventricular chamber volume, and generate an output pump control signal based on the input signal. At least one of LV end diastolic volume, end systolic volume, mean ventricular volumes, stroke volume, or ventricular volumes at any part of the cardiac cycle, or as a function can be utilized. Volumes can be weighted at different parts of the cardiac cycle differently. For example, end systolic or diastolic cardiac volumes may be weighted more. Embodiments of the system may utilize (a) constant setpoint, (b) a repeating continuous (e.g. sinusoidal) or discrete function (e.g. switching setpoint—high volume and low volume setpoints to introduce pulsatility), or a non-repeating function. The repeating setpoints can be within the same cardiac cycle or multiple cardiac cycles. The system can utilize time domain or frequency domain-based filters to filter out noise. Since the controller reads multiple coils, it has the ability to reject ones that fail. Embodiments of the system have the ability to calibrate the gain and offset of volume measurements obtained from coils periodically using echo cardiography or any imaging modalities. Ability to input the calibration factors into the control algorithm.

Suction prevention is also described. In one embodiment, the controller is configured to ensure end-systolic volumes are above a minimum setpoint. Suction detection may also include monitoring pump parameters (power) in conjunction with volume information (high frequency change in volumes) to detect ventricular suction (or atrial suction in patients with atrial inlet). Embodiments of the system can enerate pulsatility. To accomplish this, volume setpoints can be switched periodically to generate pulsatility. The pulsatility generated can be synchronous or asynchronous. Unlike the native ventricle which can generate peak flows of 30-40 L/min, the LVAD are limited to peak flow rates of ˜10 L/min which limits the level of pulsatility that can be restored. Using modulation approaches (switching of volume setpoints), the system can restore pulsatility to near physiologic levels (˜40 mmHg) at a lower frequency or modestly augment pulsatility within a cardiac cycle (synchronous modulation) within device manufacturer recommended designated minimum and maximum pump speeds or flow rates (2-10 L/min) to ensure safety.

Evaluation and estimation of cardiac function/reverse remodeling can be implemented. For example, by setting the pump speed at a low constant speed periodically and estimating the ejection fraction, rate of change of volume (contractility), ventricular end-systolic and end-diastolic volumes can provide tracking of the patient's native heart function/recovery. Automation of this function can be performed multiple times a day to negate the effect of circadian rhythm. Estimating the heart rate to ensure the patient is at rest or within a narrow range of heart rate also minimizes variability.

Opening of aortic valve (dynamically or periodically) is another aspect according to one embodiment. Aortic valve fusion is thought to occur when the aortic valve remains closed for prolonged periods of time. To mitigate this, the stroke volume (end diastolic—end systolic volumes) can be used as a setpoint that can be increased to a large value periodically (5 seconds every minute) to ensure ejection through the aortic valve. Additionally, due to circadian rhythm, the cardiac output and contractility changes during the day. Thus, the stroke volume or end systolic/diastolic volumes can be set to be such that the pump flow (can be estimated using sensorless model-based approaches) is slightly lower than the stroke volume (ensures beat to beat opening of the aortic valve)—this can also be beneficial for weaning.

For weaning, the stroke volume (or end systolic or end diastolic volume) setpoint of the native ventricle can be set such that it is in a specific range. For example, initially, the stroke volume can be set to be minimal if the patient needs maximal unloading. Subsequently, as the patient's heart condition improves, the stroke volume of the native heart can be set to be higher—this will lead to more of the workload taken by the heart (similar to rehab with any injured muscle) maximizing potential for recovery and minimize myocardial atrophy.

Exercise response can also be evaluated. The exercise response of ventricular volume and rates of change of volume can be used for evaluation of myocardial health.

### The Lumped Parameter Model of the Cardiovascular System

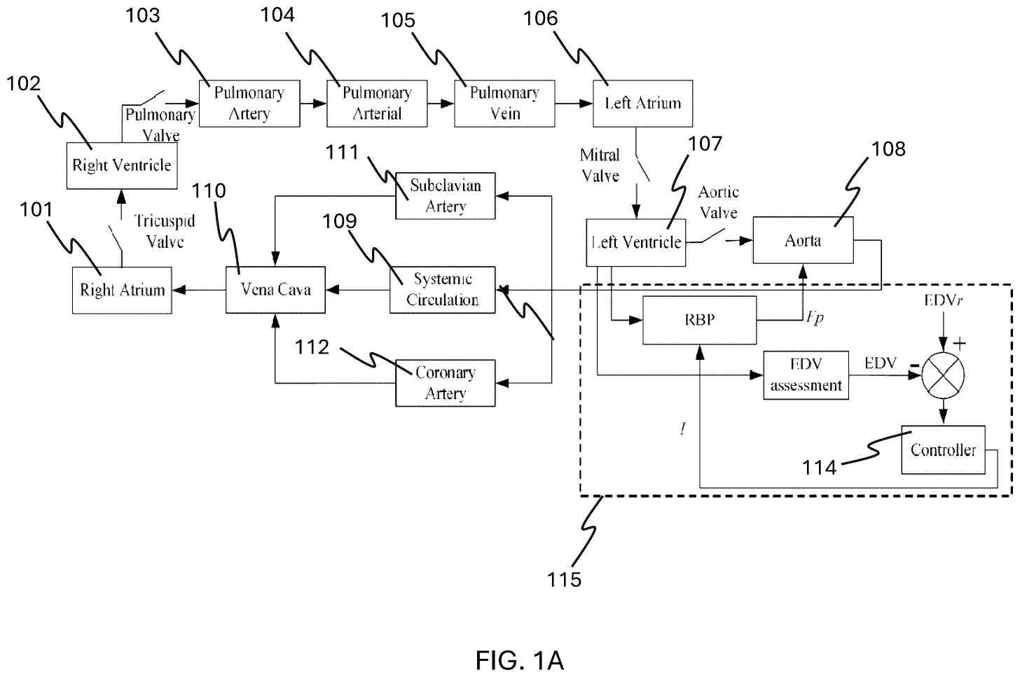

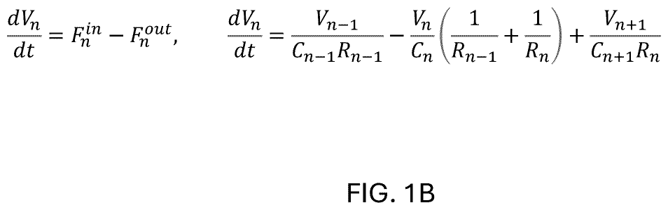

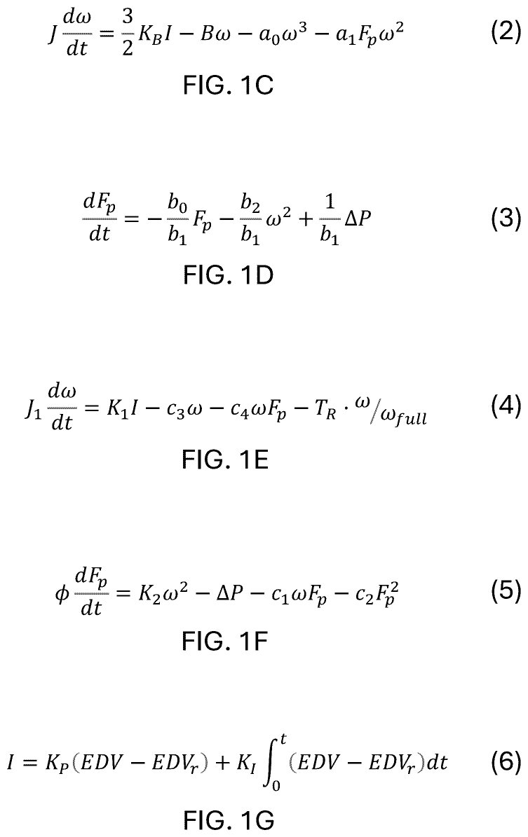

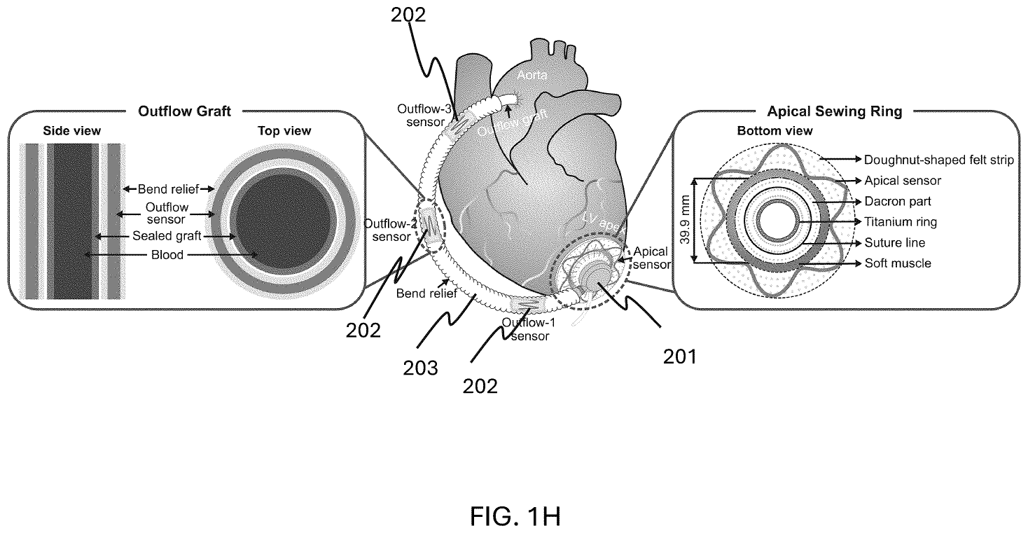

With reference now to FIG. 1A and equation (1), a nonlinear lumped parameter model (LPM) was used in this work to describe the cardiovascular system according to one embodiment. The model was repeatedly verified while developing different control strategies including end-organ perfusion, fault and suction detection, and suction prevention, etc. for various types of RBPs [38-40,42-45]. This LPM has four valves and twelve blocks FIG. 1A, valves are aortic valve, mitral valve, pulmonary valve, and tricuspid valve, while blocks are right atrium 101, right ventricle 102, pulmonary artery 103, pulmonary arterial 104, pulmonary vein 105, left atrium 106, left ventricle 107, aorta 108, systemic circulation 109, vena cava 110, subclavian artery 111, coronary artery 112. The atria and ventricles were represented using time-varying compliance (C) and resistance (R), the other eight modules were represented using constant resistance and compliance. Each block can be described using volume (V) based differential equation as shown in FIG. 1B, where in module n the dVₙ/dt represents the changing rate of V, and Fⁱⁿ and F^{out} denote blood flow into and out of module n, respectively. An axial or centrifugal RBP could be couple with the above LPM.

### Modeling of the Axial and Centrifugal RBP

With reference to FIGS. 1C and 1D, the axial RBP model was developed using differential equations (2) and (3) regarding the rotational speed and flow of the pump [50,51] according to one embodiment, where I is the control input in equation (2) as well as the pump current, ω and Fₚ are the rotational speed and flow of the axial RBP, respectively, and ΔP in equation (3) is the value of aortic pressure (AoP) minus LV pressure (LVP). The values of all the other model parameters are constant [50,51].

With reference to FIGS. 1E and 1F, the centrifugal RBP model was developed also using the differential equations regarding the rotational speed and flow of the pump as followed [52, 53], where I, ω, Fₚ, and ΔP in equations (4) and (5) are the same as those in equations (2) and (3). The values of all the other model parameters are still constant [52,53]. In FIG. 1A, RBP can for example be either the axial or centrifugal pump, which removes the blood from the LV and pumps to the aorta. The resultant hemodynamic changes affect all the twelve blocks of the entire cardiovascular system.

### Development of Pump-Independent Control Algorithm for Axial and Centrifugal RBPs

Maintaining a fixed LV end-diastolic volume (EDV) is the goal of this pump-independent control algorithm (i.e., EDV control). The EDV control strategy provides adequate end-organ perfusion and effectively prevent the occurrence of LV suction regardless of the type of RBPs. To implement the EDV control algorithm, a gain-scheduling proportional-integral (PI) controller 114 was used with the control law shown in equation (6) of FIG. 1G according to one embodiment, where EDV is the actually measured EDV value of LV, EDVr is the threshold of LV-EDV, proportional and integral coefficients of the PI controller were represented by K_{P} and K_{I}, respectively. EDVr is set as 85 ml, K_{P}/K_{I} are 0.01/0.002 for the axial RBP and 0.03/0.006 for the centrifugal RBP, respectively, and kept unchanged during all the in-silico simulations for each pump. Due to continuous unloading with LVAD resulting in a reduced preload, the end diastolic volumes are typically 60-80% of normal end diastolic volumes (˜120-130 ml). Thus, a 85 ml setpoint (70% of 120 ml) was chosen. The dashed line part of FIG. 1A (115) displays the schematic diagram of the EDV control algorithm. Embodiments of the RBP system are fully implantable, are able to transmit power across the skin/tissues to avoid driveline infection and incorporates long-term sensors that can detect and measure physiological criteria like ventricular chamber size, which can then assist in autonomously running an RBP. The RBP system and sensors are wirelessly powered to estimate chamber size based on magnetic resonance technology. With reference to FIG. 1F, embodiments of the RBP system include at least one apical sensor 201 resonantly coupled with one or more outflow sensors 202 and communicatively connected with the controller 114. Embodiments of the RBP system include at least one apical sensor 201 (resonator) incorporated within an apical sewing cuff 203 (transmitter). The size and shape of the apical sensor 201 and outflow sensors 202 can for example be based on the HeartMate 3 apical cuff and sealed outflow graft, respectively. The apical sensor is driven by the applied radio frequency power, which is wirelessly transmitted to the receivers through resonant coupling. The apical sensor 201 and the outflow sensors 202 are configured to transmit a signal to the controller 114. These resonantly coupled sensors are non-blood contacting, have low power consumption, low long term-drift (˜1 mL or 1% over 9 months), and high accuracy.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1

### In-Silico Procedure and Performance Description

The pump-independent control algorithm was tested in-silico during various steps for (1) physiological activities of rest of 80 beats per minute (bpm) and exercise of 120 bpm, (2) normal pulmonary vascular resistance (PVR) and 8-fold PVR [16,35,44,48], (3) drift of 5 ml in LV volume (80 ml setpoint, equivalent to ˜4 years of drift), and (4) EDV without and with 15% measurement noise added to left ventricular volume (LVV) through (1), (2), and (3). Performance of the proposed EDV control algorithm was also compared to the constant PS control strategy. In-silico simulation was up to 300 seconds or a minimum of 10 beats of limit cycle. Matlab (MathWorks, Natick, MA, USA) was used to analyze all the results, including AoP, mean AoP, minimum LVP, LVV, mean PF, and mean PS. All the mean values were saved and reported after the waveforms were stable. Suction was defined as the instantaneous LVP was less than 1 mmHg [38-40,42-44].

### Results

### Performance of the Pump-Independent Control Strategy Under Normal Conditions

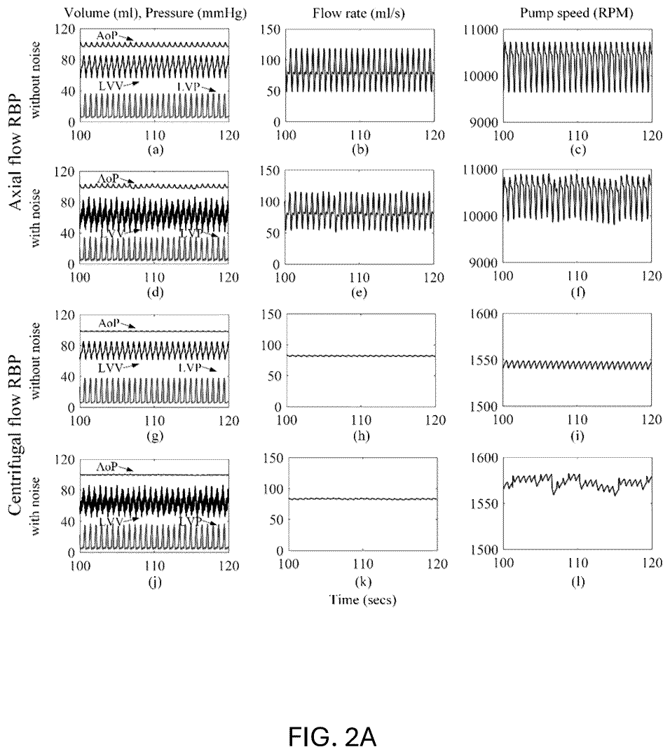

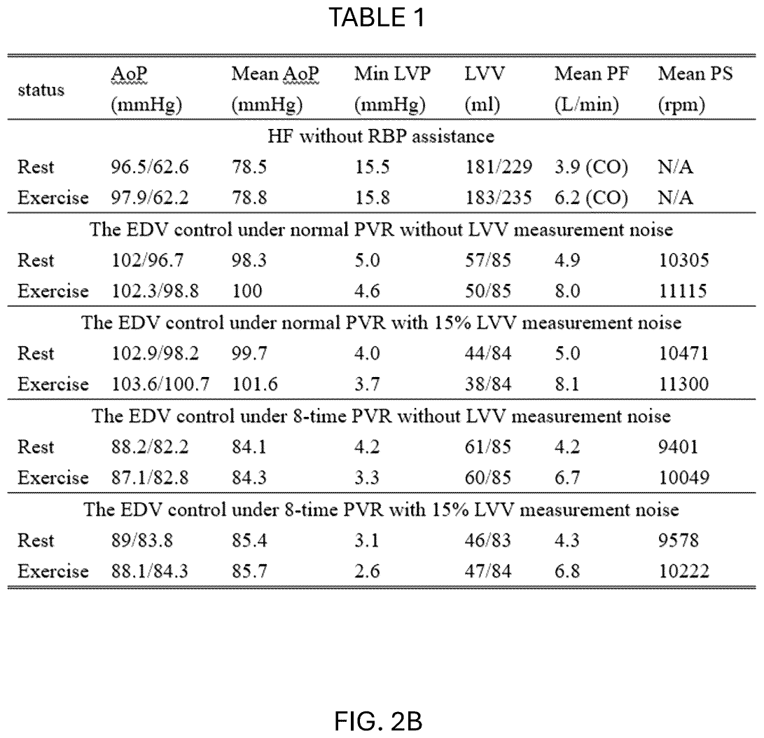

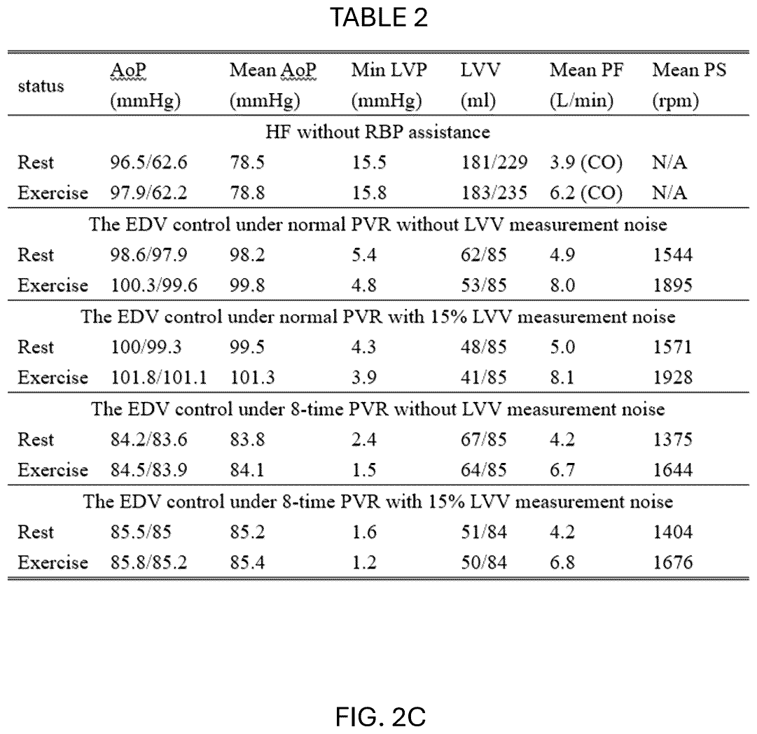

With reference now to FIG. 2A according to one embodiment, in-silico results of the pump-independent control algorithm at rest for the axial and centrifugal RBPs without and with LVV measurement noise are shown. No significant bias was observed between the performance of the pump-independent control algorithm regardless of the LVV measurement noise and RBP type (Tables 1 and 2 in FIGS. 2B and 2C respectively). The only exception is that AoP pulsatility and the variation of PF and PS under the axial RBP assistance were clearly higher than those under the centrifugal RBP assistance, but this phenomenon did not affect the EDV control strategy to produce sufficient CO and effectively prevent LV suction. Constant PS control strategy used 10471 rpm for axial RBP and 2518 rpm for centrifugal RBP, respectively. These pump speeds were obtained from the EDV control strategy at rest with 15% measurement noise. While constant PS control strategy did not cause suction events, it generated lower pump flow during exercise compared to the EDV control algorithm (Tables 1 and 2 in FIGS. 2B and 2C respectively).

### Performance of the Developed Pump-Independent Control Strategy Under the Condition of 8-Time PVR

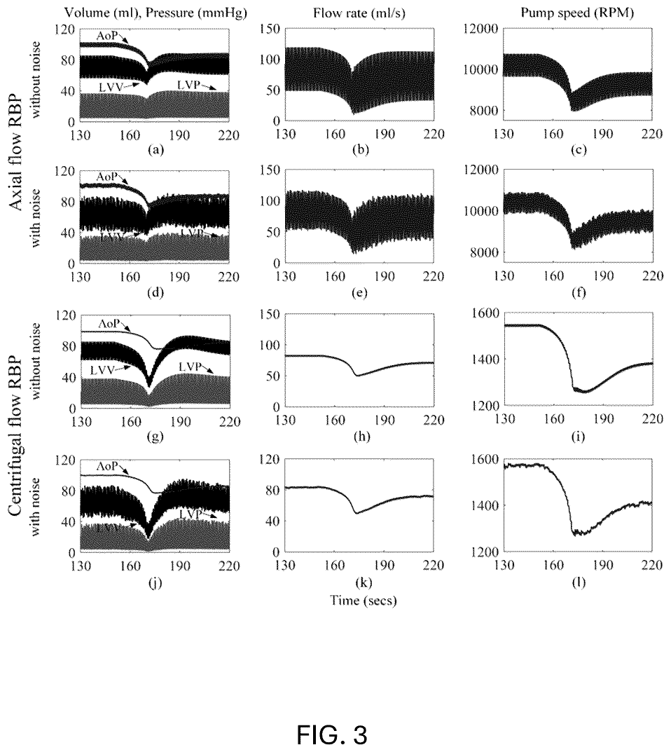

With reference now to FIG. 3, in-silico results of the pump-independent control algorithm with the rest condition of 8-time PVR for the axial and centrifugal RBPs without and with LVV measurement noise are shown. Even though there was a significant decrease in LVV and LVP during the transition from normal to 8-time PVR, LV suction still didn't happen. Additionally, the pulsations of AoP, PF, and PS with the axial RBP support were still larger than those with the centrifugal RBP support regardless of measurement noise, and the physiological perfusion under 8-time PVR condition was clearly lower than that under normal condition at rest and exercise (Tables 1 and 2) for all the cases. Furthermore, constant PS control caused intermittent suction during the PVR change at rest and had lower systolic and diastolic ventricular volumes for both pumps (Tables 1 and 2).

### Performance of the Developed Pump-Independent Control Strategy During the Transient Change

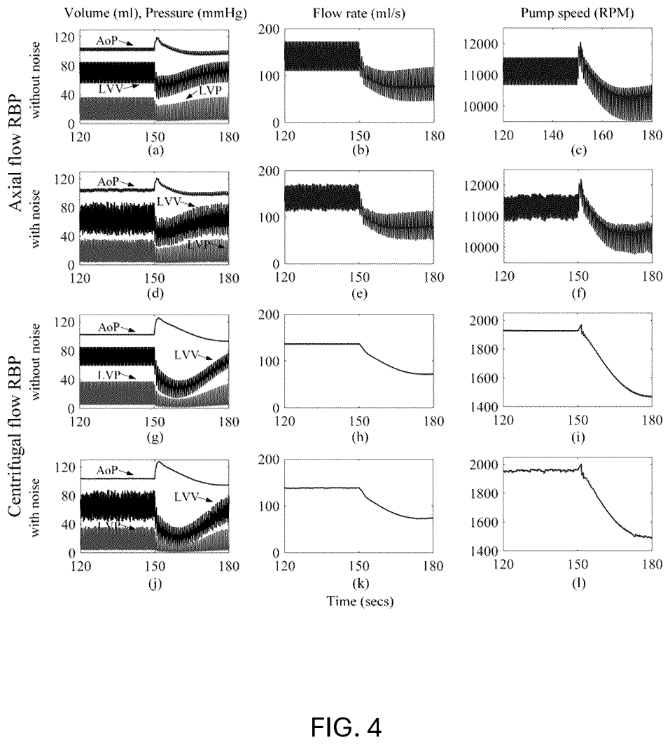

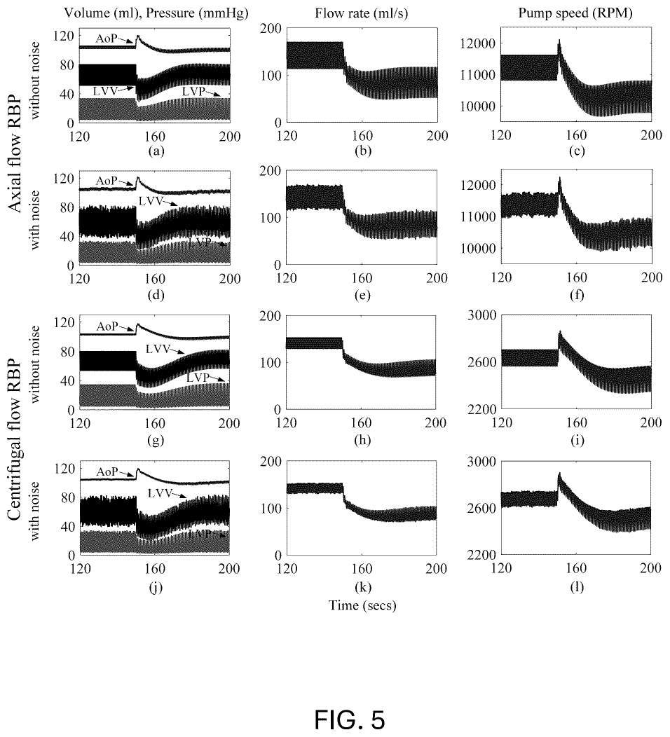

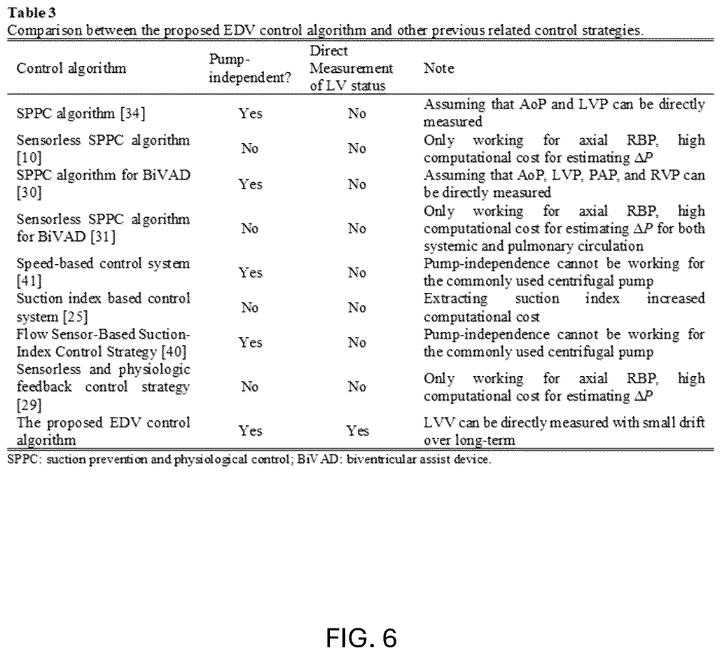

With reference to now to FIG. 4, in-silico results of the pump-independent control algorithm during the transient change from exercise to rest for the axial and centrifugal RBPs without and with LVV measurement noise are shown. The pump-independent control strategy autonomously augmented the physiological perfusion at exercise and lowered it at rest, which is higher than that without any RBP support (Tables 1 and 2). Suction was not found for all the cases. The results during the change from rest to exercise were not shown since changing the physiological condition from exercise to rest has a higher propensity for causing suction. Similarly, a 6% volume drift (5 ml) in the LV chamber size in either direction did not cause suction events and only a small increase in pump flow was observed (FIG. 5).

### Discussion

In-silico findings confirmed that the pump-independent control strategy can regulate PS to satisfy physiologic demands and at the same time effectively avoid LV suction events during various scenarios only by accurately assessing LVV with the newly designed non blood contact sensor technique. The EDV pump-independent method was successfully simulated even with some extreme states such as the permanent 8-time enhancement in PVR in 20 seconds (preload instantaneously decreased), which stands for the worse and non-physiologic case and could happen with mild coughing or Valsalva action. In addition, the transient change between rest and exercise also represented the worse and non-physiologic case. The above two cases were simulated to prove the effectiveness of the EDV pump-independent method to provide sufficient cardiac output and protection against suction.

The value of EDV is the core part of the pump-independent control algorithm. EDV could be accurately predicted using the resonantly coupled sensors [49]. These sensors were designed according to the highly sensitive relationship between the spatial separation and transmission coefficient, which stands for an effective polynomial regression. Therefore, the distance among these sensors could be effectively predicted with the accuracy of 95% without drift observed, and the size of LV chamber including EDV can be accurately evaluated. Then this study used 15% uniformly distributed noise added to LVV to test the robustness of the EDV pump-independent method, indicating that noisy LVV didn't degrade the performance of the developed EDV control strategy, which could eliminate the demand of implantable additionally indwelling pressure and/or flow sensors and enhance reliability and safety of the RBP system.

The EDV control strategy synchronizes the support of LV provided by the axial and centrifugal RBPs to the native control mechanisms of the circulatory system. For instance, because of Frank-Starling mechanism, the increased venous return from rest to exercise will generate an increase in LV contractility with a higher preload. The augmented native LV contractility results in a higher PF to maintain the desired EDV threshold and satisfy the CO. Similarly, a decreased native LV contractility due to the reduction of physiological demands can cause less PF to prevent LV suction events. Thus, the pump-independent control strategy was sensitive to the LV chamber size, but not sensitive to the heart rates regardless of the type of RBPs.

Axial and centrifugal RBPs were used in these experimental examples. The pump fluid flow of the axial RBP is along the direction of the axis, usually vertically into and horizontally out of the direction, while the fluid flow of the centrifugal RBP is from the center to the periphery, that is, along the direction of the centrifugal force. Except for the fluid flow direction, the other main differences between the axial and centrifugal RBPs also lie in their application scope, efficiency, and shape structure, etc. Admittedly, differences in the AoP pulsatility and variation of PF and PS as the pump dynamics between the axial and centrifugal RBPs were found in FIGS. 2A-4 and Tables 1 and 2, and HQ curves can also indicate that the two types of RBPs have intrinsically different sensitivity to AP in equations (3) and (5). However, the developed EDV control strategy generated similar tested findings for axial and centrifugal RBPs, which testified pump independence of the algorithm.

Both axial and centrifugal pumps provide physiologic levels of flow to unload the ventricle (2-10 L/min) The dynamics and performance curves of these pumps vary, but the performances are similar. This is reflected in the similar steady state results for both axial and centrifugal pumps in Tables 1 and 2 and the pump-independent control algorithm. The differences in the pump dynamics are reflected in the transitions as shown in FIGS. 3 and 4. Maintaining LV chamber size emulates Frank-Starling mechanism and is agnostic of the type of pump, leading to a pump-independent control scheme. The pump-independent control algorithm synchronizes the support of LV provided by the axial and centrifugal RBP to the native control mechanisms of the circulatory system and prevents the ventricle from experiencing volume overload. For example, as the venous return increases due to increased physical activity, the pump-independent control algorithm increases pump speed to maintain LV chamber size. Similarly, when the venous return is reduced, the pump-independent control algorithm reduces the pump speed to avoid suction. Suction was avoided even with very rapid worst-case scenarios including transition from rest to exercise and an abrupt reduction in venous return.

The pump-independent control algorithm relies on a direct measure of ventricular volume load, increasing the preload and afterload sensitivity of the RBP. These sensors were designed and tuned according to the highly sensitive relationship between the spatial separation and transmission coefficient, using a polynomial regression. Therefore, the distance among these sensors could be effectively predicted with a high degree of accuracy. This study used 15% uniformly distributed noise added to LVV to test the robustness of the proposed EDV pump-independent method, indicating that noisy LVV didn't degrade the performance of the developed EDV control strategy. Significantly, the novel sensor technology has minimal long-term drift (˜1% in 9 months of use). Physiologic control and suction prevention were achieved even with ˜4 years of cumulative drift, demonstrating the robustness of the pump-independent control algorithm. The LV chamber size estimation can be calibrated using echocardiography when a patient goes in for a checkup every few months. Furthermore, unlike other technologies to measure pressure and volume, the resonantly coupled sensors are non-blood contacting and are not affected by tissue ingrowth. They are designed to fit on the apical cuff and outflow graft of RBP, have low power consumption, and are ideal for long-term measurement. Even with a loss of one of the receivers, the sensors can still estimate LV chamber size, providing sensor redundancy. Importantly, the resonantly coupled sensors can be used in conjunction with wireless power transmission to the RBP using TETS, eliminating driveline infection. Table 3 in FIG. 5 compares the pump-independent control algorithm to previous related work. The pump-independent control algorithm uses ventricular volume which is a direct measurement of ventricular status, regardless of the pump used, while the previous algorithms relied on pump parameters and indirect measurement of ventricular status.

The controller proportional and integral gains are different for each pump but can be tuned a priori using in-silico or in-vitro methods. There are differences in the pump performance and dynamics of each pump, leading to differences in pump speed and hemodynamic pressure variations, despite similar pump flows. Controlling the EDV ensures that the ventricle is not volume overloaded, while providing physiologic perfusion. In addition to providing physiologic control, the LV chamber size estimation can be used as a diagnostic tool to measure the contractility of the heart. For example, the RBP can be run at a low speed and the LV contractility can be estimated using the rate of change of chamber size. Repeated periodically over long implant durations, this type of estimation can lead to long term monitoring of reverse remodeling of the heart. When warranted, the control algorithm can be tuned to ensure increasing LV stroke and end-diastolic volume, enabling weaning from the pump.

The in-silico model limitations include instantaneous valves, Newtonian blood, lack of Baroreflex mechanism, and neglects of the effects of gravity. Elimination of these limitations would better represent the relationship between the RBP and cardiovascular system. It is the standard practice to use in-silico models with these limitations for the development of control algorithms. Despite these limitations, the in-silico model demonstrated the feasibility of the pump-independent control algorithm. The drift and noise characteristics of the LV chamber size estimation were derived from in-vitro data. Mock circulation and animal studies will be used to further validate the performance of the pump-independent control algorithm. While the pump-independent control algorithm can prevent suction due to LV chamber size reduction, it cannot prevent local suction due to highly suboptimal RBP inflow cannula angle.

This study still confirmed the feasibility of the EDV pump-independent method and provided meaningful prospects in the feedback control algorithm.

Embodiments of a pump-independent control algorithm for the axial and centrifugal RBPs described herein can be utilized for end-organ perfusion and avoidance of LV suction. The core part of this pump-independent control algorithm is based on the EDV value. Simulation results shown that the EDV control strategy effectively provided adequate CO, and in the meantime successfully avoided occurrence of LV suction regardless of the LVV measurement noise or the type of RBPs. The EDV method is pump-independent and could be coupled with the control systems for the axial and centrifugal RBPs. The pump-independent control algorithm can provide physiologic perfusion by augmenting CO during exercise and avoid suction by reducing pump speed with diminished venous return. The pump-independent control algorithm was robust even with 6% drift and minimal performance degradation was observed with 15% measurement noise. The control algorithm is pump-independent, non-blood contacting and is not impacted by tissue ingrowth.

### Example 2

### A New Pump-Independent Algorithm of Physiological Control and Suction Prevention Based on End-Systolic Volume for the Axial and Centrifugal Flow Pumps

Continuous-flow left ventricular assist devices (CF-LVADs) are a cornerstone therapy for end-stage heart failure but face limitations due to fixed-speed operation, which compromises physiological adaptability and pulsatility, increasing risks of suction, vascular complications, and end-organ hypoperfusion. A pump-independent control algorithm is described using end-systolic volume (ESV) as a feedback parameter, enabled by a non-blood-contact ventricular volume sensor with minimal drift over chronic use. The pump-independent control algorithm integrates a gain-scheduling proportional-integral (PI) controller to maintain ESV at 40 mL, a pulsatility induction strategy modulating ESV setpoints, and a safety mode to prevent suction during abrupt hemodynamic shifts. Validated in-silico using a 16-element cardiovascular model, the strategy demonstrated robust performance across axial and centrifugal LVADs under rest, exercise, 8-fold pulmonary vascular resistance (PVR) increases, and 6% ventricular volume measurement noise. Key outcomes included sufficient cardiac output (5.0-8.5 L/min), physiological mean aortic pressure (83-104 mmHg), and restored pulse pressures (23-40 mmHg) with 64% ejection fraction—24% higher than non-pulsatile operation. The safety mode prevented suction during exercise-to-rest transitions and extreme PVR scenarios, while centrifugal LVADs exhibited superior noise resilience. Despite subphysiological pulsatility frequencies (2-10 cycles/min), the algorithm reduced the mean pump speeds, reducing the shear stress on the blood cells and enhancing biocompatibility and battery life. Limitations include the model's simplified physiological assumptions (Newtonian fluid, idealized valves). Future work requires in vivo validation and pulsatility optimization to align with autonomic rhythms.

Left ventricular assist devices (LVADs) are currently an important therapy for end-stage heart failure (HF), where they serve as both bridge-to-transplant and long-term destination therapy due to the global shortage of donor hearts [54, 55]. These devices are surgically implanted to augment cardiac output (CO) and reduce the workload of the failing ventricle to provide life-saving mechanical support. Continuous flow LVADs (CF-LVADs) have become well-accepted clinically because they are small, minimally invasive, and durable compared to previous generations of pulsatile devices, which effectively decreased surgical injury, electrical power consumption and risk of thrombosis, and improved mobility for patients [56, 57].

However, despite their clinical acceptance, CF-LVADs present several limitations due to their typical fixed-speed operation. This fixed speed mode presents challenges in adapting to the variable physiological demands of patients like rest and exercise [58]. Additionally, the inability to dynamically adjust the pump speed poses difficulties in preventing left ventricular (LV) suction, maintaining optimal perfusion, and preventing percutaneous site infections [58]. For example: If the pump speed doesn't match the required flow by the body, Ventricular suction which leads to LV wall collapse and myocardium damages would happen when the flow is high [59]. In contrast, for low pump speed; low flow rate, the LV is overloaded which causes more tension on the LV walls [60].

To address these issues, a diverse range of control algorithms have been developed to optimize LVAD performance and meet physiological demands. Existing literature reports the utilization of control algorithms relying on left ventricle (LV) pressure, aortic pressure, pump inlet and outlet pressures, measured pump flow or heart rate [59, 61-65]. The utilization of control algorithms that rely on Left Ventricular (LV) pressure as an indicator to regulate blood flow necessitates the installation of a pressure sensor at the LVAD inlet for systolic pressure measurement. This sensor signal is then employed to modulate the LVAD speed. While these LV pressure control algorithms facilitate physiological adaptation to varying perfusion requirements, the incorporation of an inlet pressure sensor introduces certain limitations, such as sensor error, the risk of thrombosis and potential drift over time [62]. In some instances, the pressure sensor drift can reach up to ±25 mmHg [62], requiring regular calibration for offset adjustments. The lack of long-term stability of these sensors hinders their clinical application as physiological control algorithms, as the LVADS are supposed to have a lifetime of 10-20 years [66]. The application of heart rate as a control parameter is limited, as it is incompatible with patients exhibiting severe arrhythmia and may lead to ventricular collapse in certain cases [65]. Sensorless control strategies such as employing motor current or fuzzy logic controllers can eliminate the drawbacks of inserting unreliable sensors [54]. However, these approaches face their own limitations. For example, the motor current control algorithms cannot consistently maintain adequate perfusion across diverse clinical conditions and physical activities, and the fuzzy logic control algorithms assume linear relation between the flow and the heart rate which is not the case in a wide range of physical activities [67].

In addition to these control limitations, CF-LVADs pose physiological concerns due to their diminished arterial pulsatility. The fixed-speed operation significantly reduces pulsatility in the arterial system, which has been associated with numerous adverse clinical outcomes, such as aortic valve fusion, aortic insufficiency, gastrointestinal bleeding, hemorrhagic stroke, and the development of arteriovenous malformations. It has also been linked to acquired von Willebrand syndrome, a bleeding disorder exacerbated by low pulse pressure [68, 69]. Moreover, prolonged exposure to non-pulsatile hemodynamics can result in vascular remodeling, including thickening and stiffening of the aortic wall, impaired endothelial function, and loss of smooth muscle cells [70]. Structural changes such as adventitial fibrosis have also been documented in the coronary vasculature under reduced pulsatility conditions [71]. Clinical data further suggest that gastrointestinal bleeding is strongly associated with pulse pressures below 35 mmHg [72]. Efforts to reintroduce pulsatility through speed modulation have been implemented in commercial devices, but remain insufficient. Devices like the Jarvik 2000 device and HeartWare device introduced periodic speed modulation to restore pulsatility, but their effects were limited due to infrequent adjustments [73]. The HeartMate III device improved on this with speed changes every 2 seconds, yet it still produces only modest pulse pressures of 10-20 mmHg, which is insufficient to prevent many of the complications [73].

In response to these limitations, a pump-independent control algorithm is described using a validated non-blood-contact ventricular volume sensor to estimate end-systolic volume (ESV) as the physiological feedback signal for LVAD control. This sensor has demonstrated reliable long-term performance with minimal drift over a three-month evaluation period, making it suitable for chronic use in implantable devices [74]. To counteract the loss of pulsatility commonly seen in continuous-flow LVADs, the algorithm introduces periodic modulation of the ESV setpoint to restore arterial pulse pressure. Furthermore, the control algorithm incorporates a safety mode to handle sudden hemodynamic changes, providing a universal and adaptable framework for axial and centrifugal LVAD designs.

### Methods

### The Cardiovascular System Lumped-Parameter Model

A sixteen-element lumped-parameter model was utilized to represent the human cardiovascular system. This model has been used to test and validate various control algorithms for different rotary blood pumps such as LVADs, cavopulmonary assist devices, and bi-ventricular assist devices [75-80]. This model consists of twelve blocks and four valves, as shown for example in FIG. 1A. The blocks correspond to the major components of the circulation system: the heart, the systemic and venous circulation, and the pulmonary circulation. The heart is represented by four blocks: the left and right ventricles and atria. The systemic circulation is modeled by a five block as follows: aorta, subclavian artery, coronary artery, other systemic arteries, and vena cava. Three blocks represent the pulmonary circulation: the pulmonary artery, pulmonary arterioles, and pulmonary veins. The four valves-tricuspid, pulmonary, mitral, and aortic valves-are modeled as time-dependent resistances that vary during systole and diastole in each cardiac cycle [80].

All blocks in the model are treated as static (non-time-dependent) blocks, except for the heart blocks and the coronary artery, which are considered “active” blocks. These active blocks are characterized by time-varying compliance to reflect the changes in myocardial compliance during systole and diastole. Each block is represented by a first-order differential equation (Equation 1 depicted in FIG. 1B), that relates the volume (V), compliance (C), and resistance (R) of the respective block where dVₙ/dt is the change of volume over time in block n, and Fₙⁱⁿ and Fₙ^{out} are the blood flow rates into and out of the block, respectively.

### Modeling of the LVAD

To evaluate the independence of the control algorithm from the type of LVAD blood flow, two different LVAD types—axial flow and centrifugal flow—were used in this study. The LVAD was introduced in the cardiovascular model in parallel with the aortic valve, connecting the left ventricle to the aorta (FIG. 1A). This introduction of the LVAD mainly affected the equations governing the LV and aorta blocks. Each LVAD type was represented in the model by two first order differential equations: one describing motor speed (Equation 2 depicted in FIG. 1C) and the other governing pump flow rate (Equation 3 depicted in FIG. 1D), where J is the motor polar moment of inertia, I is the electric current and it is the control variable in the control algorithm, ω and Fₚ are the speed and flow rate of the LVAD, respectively, and ΔP is the difference between aortic pressure (AoP) and LV pressure (LVP). K_{B}, B, a₀, a₁, b₀, b₁, and b₂ are correlation and experimental constants detailed in [81, 82].

For the centrifugal flow LVAD, the differential equations developed by Petrou et al. [83], were employed to represent pump performance (Equation 4 depicted in FIG. 1E and Equation 5 depicted in FIG. 1F), where I, ω, Fₚ, and ΔP in equations (4) and (5) are the same as those in equations (2) and (3) and the values of constants J₁, K₁, c₁, c₂, c₃, c₄, T_{R} and ω_{full} are detailed in [83].

### The Controller for the Axial and Centrifugal LVADs

The primary purpose of the pump-independent control algorithm is to maintain the left ventricle end systolic volume (ESV) setpoint(s). This pump-independent control algorithm is designed to prevent suction events in the LV during rapid shifts between different physiologic demands or pulmonary vascular resistance. Additionally, the algorithm aims to ensure adequate CO and blood pressure for proper physiologic perfusion under different activity levels.

To achieve these goals, a gain-scheduling proportional-integral (PI) controller is implemented in the proposed ESV control strategy. The PI controller regulates the electric current (I) supplied to the pump—used as the control input—based on the error between the reference and measured ESV values (FIG. 1A). The control law is expressed by Equation 6 (FIG. 1G), where I is the pump electric current, ESVₘ is the measured ESV value of LV, ESVᵣ is the setpoint of the PI controller, and K_{P} and K_{I} are the proportional and integral gain values, respectively. The ESVᵣ is set at 40 ml for both LVAD types. However, during the pulsatility study, the ESVᵣ is periodically modulated between high and low values to introduce pulsatile behavior into the control response. The K_{P} and K_{I} are set 0.01 and 0.002, respectively, for the axial flow LVAD and 0.065 and 0.007, respectively, for the centrifugal flow LVAD. The gain values are kept fixed throughout all the in-silico experiments, including the pulsatility study.

### In-Silico Procedure and Performance Description

First, simulations were conducted without LVAD to demonstrate the baseline values at rest with heart rate (HR) of 80 beats per minute (bpm) and exercise with HR of 120 bpm and reduced vascular resistance [84]. These baseline values provided a reference for evaluating the impact of LVAD introduction and the associated control algorithm on physiological outcomes. Subsequently, in-silico simulations were performed during various physiological scenarios to test the efficiency and robustness of the LVADs' control algorithm. These scenarios are (1) rest and exercise using the same HR values as mentioned above, (2) gradual increase, over 20 seconds, in the pulmonary vascular resistance from normal to 8-fold for both rest and exercise conditions, and (3) sudden change from exercise to rest. In-silico time lasts up to 400 seconds. To assess the robustness of the control algorithm, all simulations were repeated with and without the addition of 6% measurement noise added to the left ventricular volume (LVV) signal. MATLAB (MathWorks, Natick, MA, USA) was used to analyze all the results, including AoP, mean AoP, minimum LVP, LVV, aortic and pump flow rates, and pump current and speed. All these values were saved and reported after the waveforms were stable. Suction was defined as the instantaneous LVP was less than 1 mmHg [85, 86].

### Safety Control Mode for Suction Prevention

A safe mode was implemented to mitigate the risk of LV suction during abrupt physiological transitions. When the LVV drops below a predefined threshold of 30 mL, the current drops to a predetermined value for a duration of 5 seconds or until LVV rises above the threshold. This safety mechanism may be critical during scenarios such as sudden shifts from exercise to rest or rapid increases in PVR to eightfold. The safe mode helps prevent suction events, which could otherwise lead to pump thrombosis and hypoperfusion-induced mortality.

### Pulsatility Induction Strategy

Since continuous-flow LVADs inherently lack pulsatility, which can lead to physiological drawbacks, we introduced a pulsatility mode to the controller to enhance vascular pulsatility. This was achieved by dynamically alternating the ESVᵣ in the PI controller based on the measured LVV. Specifically, when the measured LVV exceeds 110 mL, the ESVᵣ is set to a low value of 35 mL (ESVLr), promoting ventricular unloading. Conversely, when LVV falls below 40 mL, the ESVᵣ is switched to a high value of 100 mL (ESV_{Hr}), allowing for enhanced ventricular filling.

### Results

### Performance of the Cardiovascular System Lumped-Parameter Model Without LVAD Under Normal Conditions

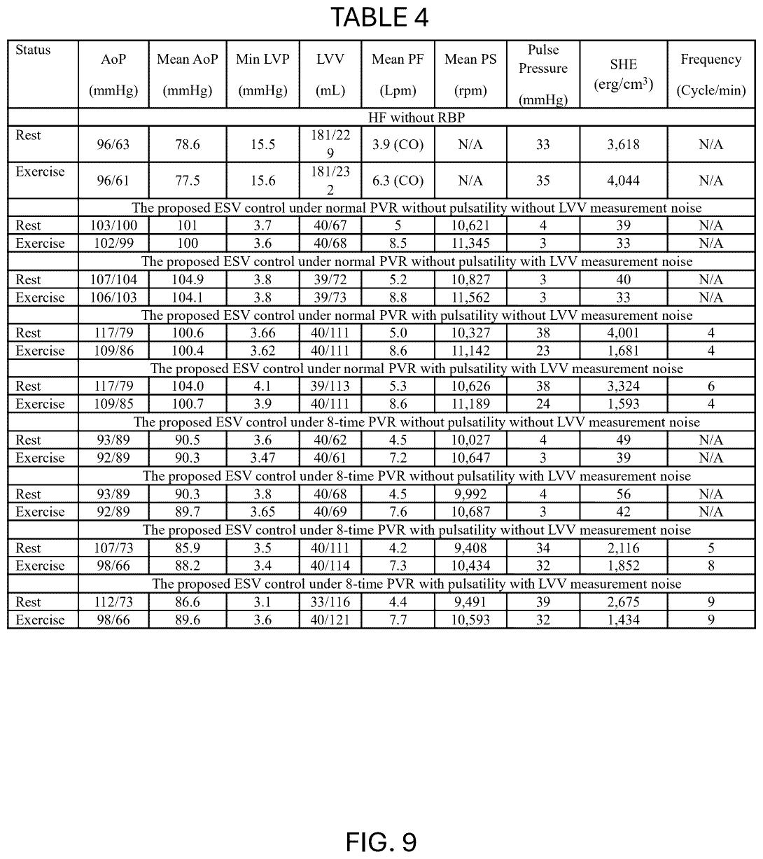

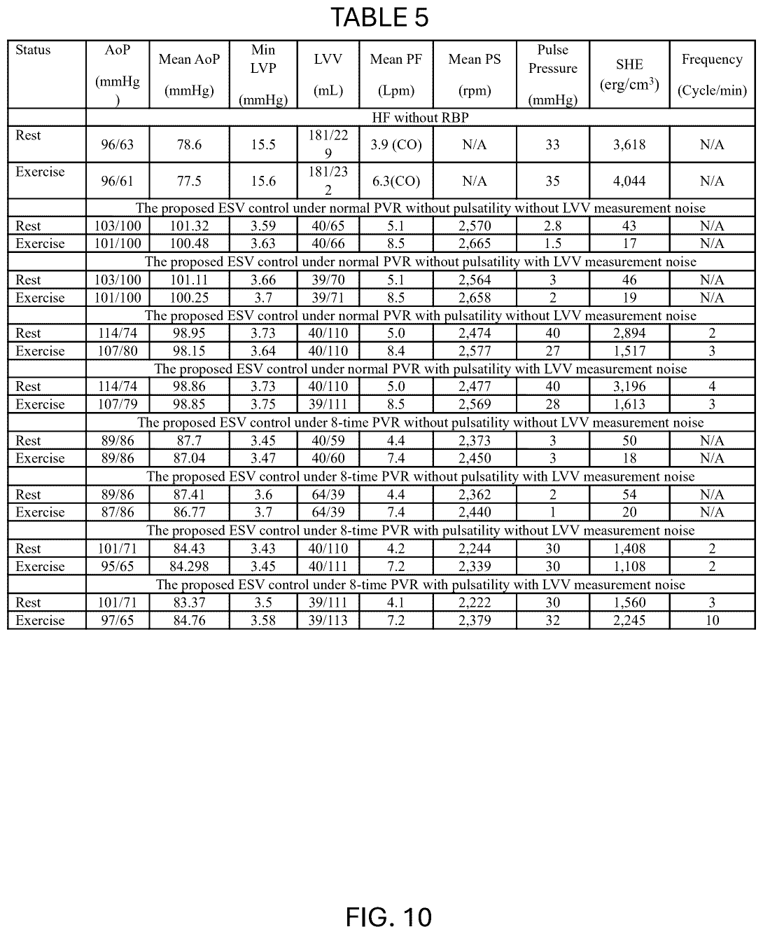

The baseline performance of the cardiovascular model without an LVAD was assessed under both resting and exercise conditions, Table 4 (FIG. 9) and Table 5 (FIG. 10) for axial and centrifugal flow LVADs respectively. At rest, the AoP was measured at 96/63 mmHg, with a mean AoP of 78.6 mmHg. The minimum LV pressure (Min LVP) was 15.5 mmHg, and the LVV ranged between 181 mL and 229 mL. The cardiac output (CO) was 3.9 LPM, and the pulsatility was 33 mmHg. The surplus hemodynamic energy (SHE) was 3618. During exercise, the AoP slightly decreased to 96/61 mmHg, while the mean AoP decreased to 77.8 mmHg. The LVV increased to 181/232 mL, and the cardiac output rose to 6.27 LPM, indicating the body's higher demand for perfusion during exercise. Pulsatility increased to 35 mmHg, and the SHE increased to 4043. These baseline values were used as a reference for evaluating the impact of LVAD support in subsequent simulations.

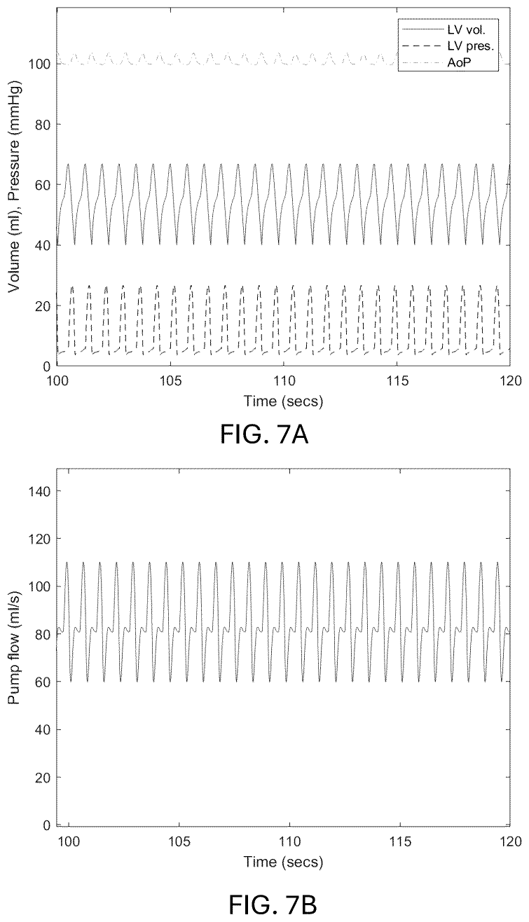

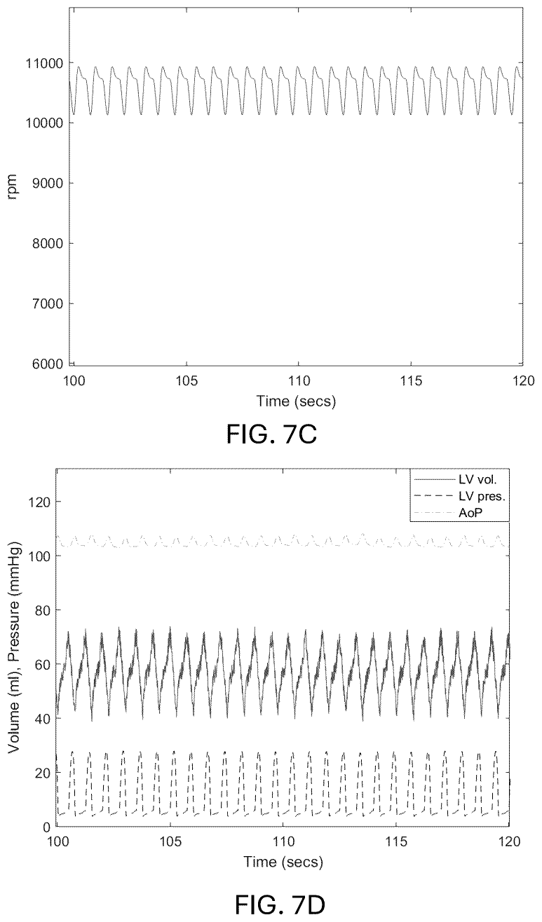

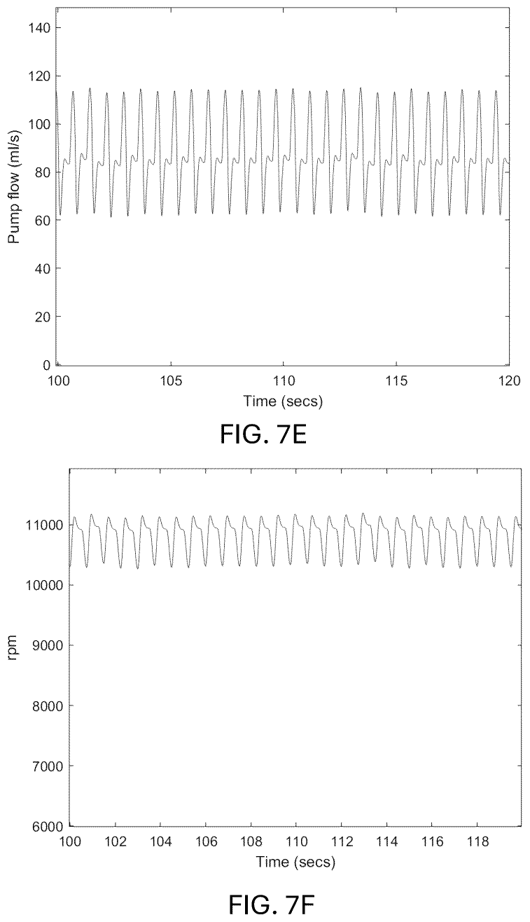

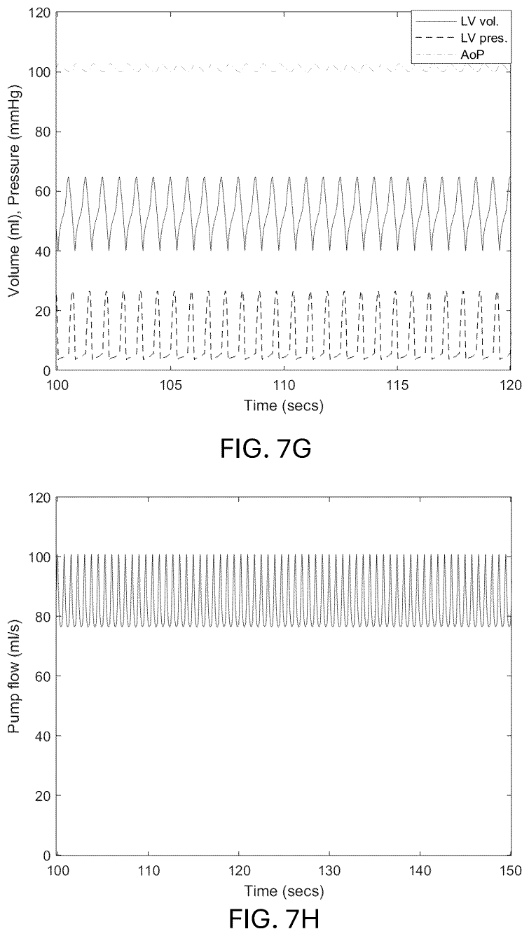

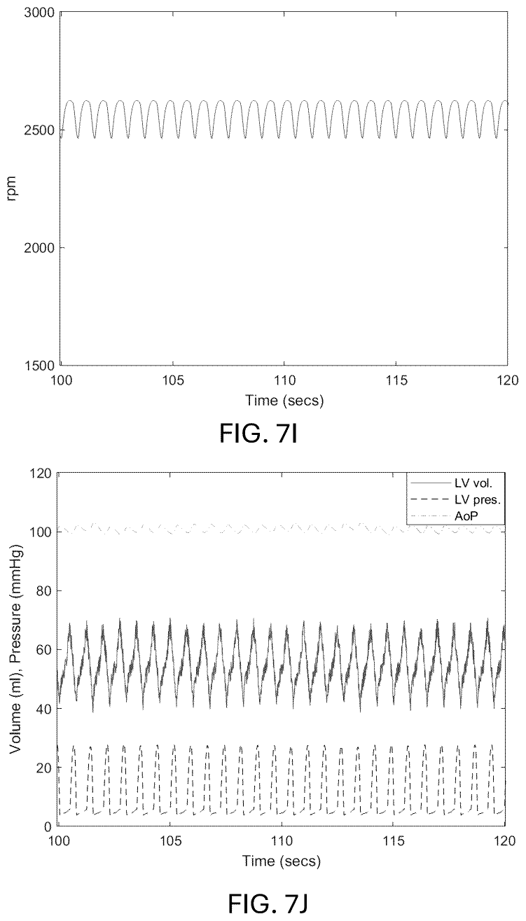

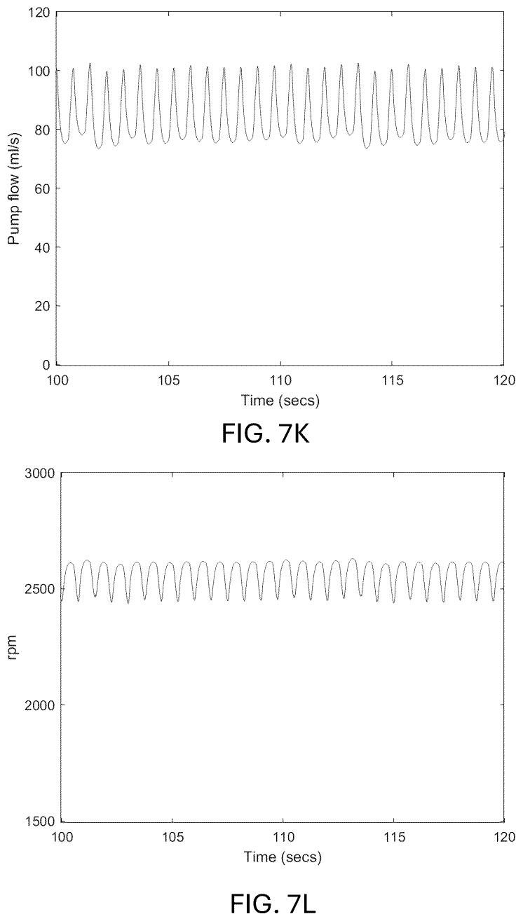

FIGS. 7A-7L depict in-silico results of the pump-independent control strategy without pulsatility at rest. FIGS. 7A-7C depict in-silico results for the axial RBP without LVV measurement noise. FIGS. 7D-7F depict in-silico results for the axial RBP with LVV measurement noise. FIGS. 7G-7I depict in-silico results for the centrifugal RBP without LVV measurement noise. FIGS. 7J-7L depict in-silico results for the centrifugal RBP with LVV measurement noise. Table 4 (FIG. 9) shows results of the pump-independent EDV control algorithm for the axial flow LVAD under various physiological conditions, with and without 6% LVV measurement noise. Table 5 (FIG. 10) shows results of the pump-independent EDV control algorithm for the centrifugal flow LVAD under various physiological conditions, with and without 6% LVV measurement noise.

### Performance of the Pump-Independent Control Strategy Under Normal Rest and Exercise Conditions With Constant ESV Set Point Without Pulsatility

With fixed ESVᵣ control strategy without pulsatility, to ensure a fair comparison, ESVᵣ=40 mL was selected as it was able to provide sufficient pump flow of 5.0 and 8.5 LPM for the axial flow LVAD and 5.1 and 8.5 LPM for centrifugal flow LVAD at rest and exercise without measurement noise, respectively, compared to 3.9 and 6.3 LPM for HF without LVAD at rest and exercise, respectively (Table 4 (FIG. 9) and Table 5 (FIG. 10)). Also, the mean AoP was increased to 101 and 100 mmHg for rest and exercise, respectively, for both axial and centrifugal flow LVADs, compared to the baseline without using LVAD (Tables 4 and 5, shown in FIGS. 9 and 10, respectively). But the pulse pressure was diminished to 4 and 2.78 mmHg for Axial and Centrifugal LVADs at rest, respectively. In addition, the SHE at rest was reduced very much by around 99% lower than the baseline for both LVADs. During exercise, the pulse pressure reduced to 3 and 1.5 mmHg for Axial and Centrifugal LVADs, respectively. And the SHE was reduced especially in the centrifugal flow LVAD to 16.8 erg/cm3. No significant bias was observed between the performance of the developed pump-independent control algorithm regardless of the LVV measurement noise and LVAD type, (Tables 4 and 5, shown in FIGS. 9 and 10, respectively). The only exception is that AoP pulsatility and the variation of PF and PS under the axial LVAD assistance, but this phenomenon did not affect the proposed ESV control strategy to produce sufficient CO and effectively prevent LV suction.

### Performance of the Developed Control Strategy Under Normal Rest and Exercise Conditions With Pulsatility

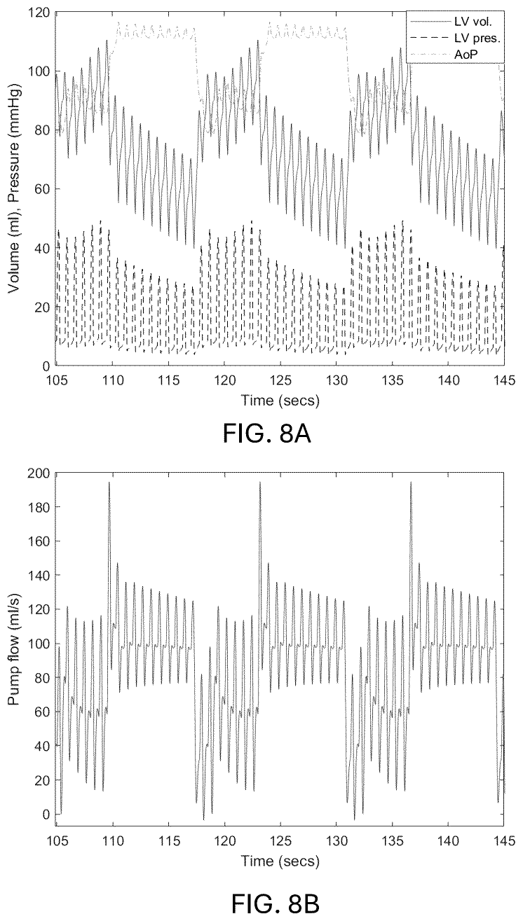

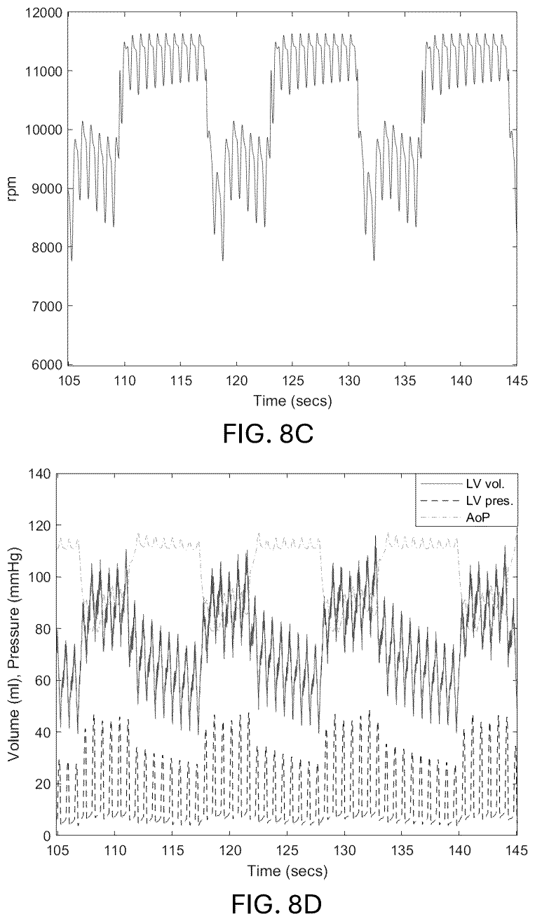

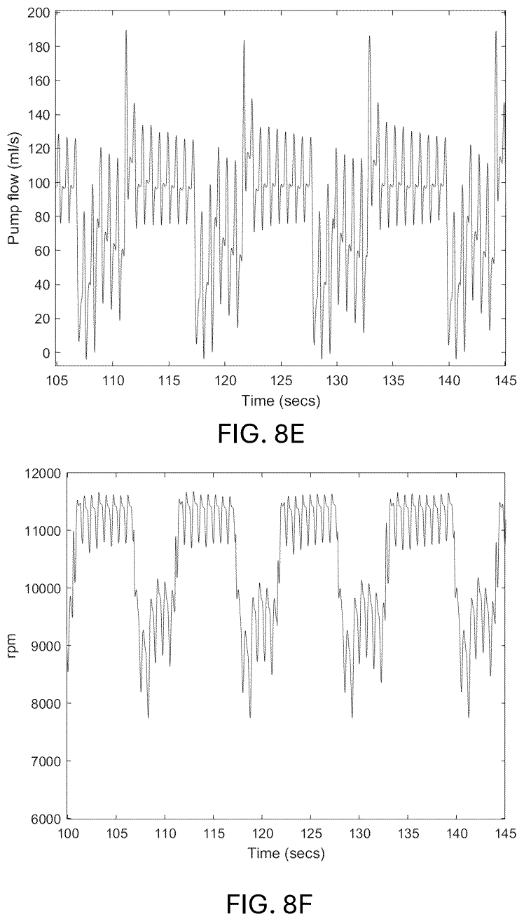

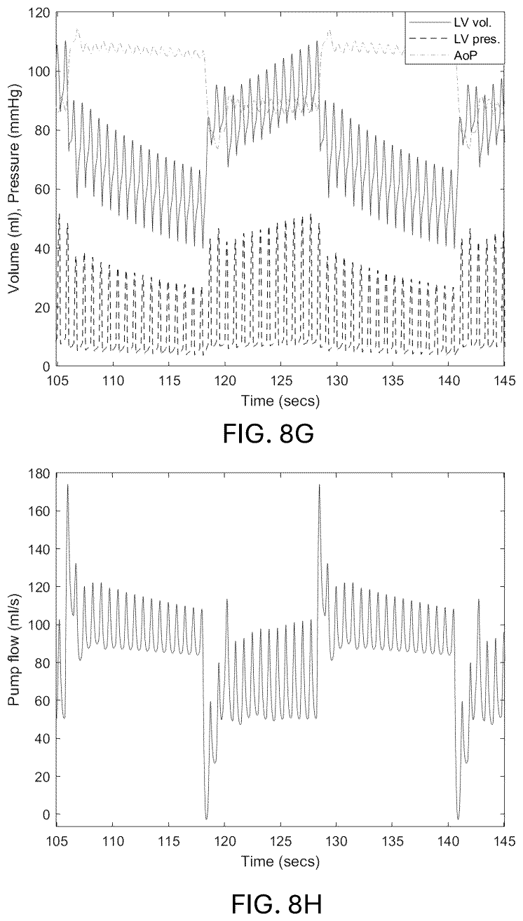

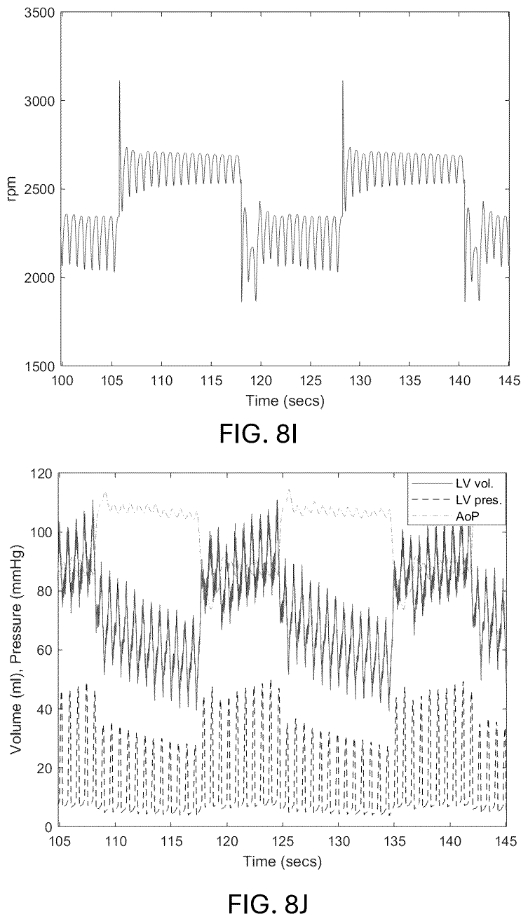

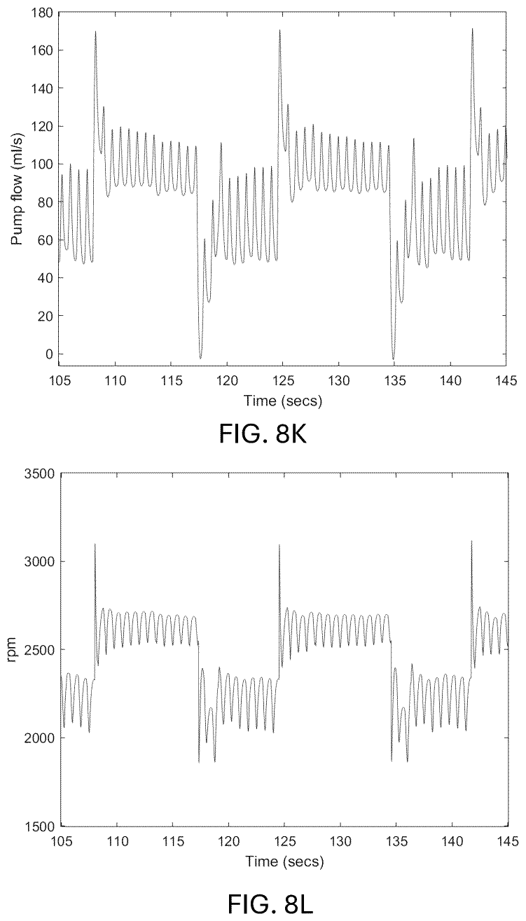

FIGS. 8A-8L illustrate the performance of the pump-independent control algorithm operating in pulsatility mode for both axial and centrifugal LVADs at rest, with and without LVV measurement noise. FIGS. 8A-8L depict in-silico results of the pump-independent control strategy with pulsatility at rest. FIGS. 8A-8C depict in-silico results for the axial flow LVAD without LVV measurement noise. FIGS. 8D-8F depict in-silico results for the axial flow LVAD with LVV measurement noise. FIGS. 8G-8I depict in-silico results for the centrifugal flow LVAD without LVV measurement noise. FIGS. 8J-8L depict in-silico results for the centrifugal flow LVAD with LVV measurement noise.

For the axial flow LVAD at rest without noise, the maximum, minimum, and mean aortic pressures were 117, 79, and 101 mmHg, respectively, resulting in a pulse pressure of 38 mmHg. The pulsatility frequency was 4 cycles per minute, and the specific hydraulic energy (SHE) was 4001 erg/cm³, while maintaining a mean pump flow (PF) of 5.0 L/min. During exercise, the controller maintained a pulse pressure of 23 mmHg and the same pulsatility frequency of 4 cycles per minute. In both rest and exercise conditions, the LVV varied between 40 and 111 mL, yielding a 64% ejection fraction-an improvement of 24% compared to the control strategy without pulsatility, as shown in Table 5 (FIG. 10).

Under elevated PVR conditions (8-fold increase), the axial pump demonstrated reductions in mean PF and in aortic pressure metrics compared to normal PVR with pulsatility. However, the pulsatility frequency increased to 5 and 8 cycles per minute during rest and exercise, respectively. Pulse pressure and SHE decreased at rest but were elevated during exercise relative to the non-pulsatile control mode.

For the centrifugal LVAD, the maximum, minimum, and mean aortic pressures were 114, 74, and 99 mmHg, respectively, yielding a pulse pressure of 40 mmHg. The pulsatility frequency was 2 cycles per minute, and the SHE was 2895 erg/cm³, while maintaining a mean PF of 5.0 L/min. During exercise, the controller preserved a pulse pressure of 27 mmHg and a pulsatility frequency of 3 cycles per minute. Similar to the axial pump, the LVV ranged from 40 to 110 mL, with a corresponding 64% ejection fraction, representing a 24% increase compared to the non-pulsatile control, as reported in Table 5 (FIG. 10).

With elevated PVR, the centrifugal pump exhibited reduced mean PF and aortic pressures relative to the normal PVR condition. Pulsatility frequency remained constant at rest (2 cycles/min) but increased to 3 cycles/min during exercise. Pulse pressure and SHE were reduced at rest, whereas pulse pressure remained unchanged during exercise compared to the non-pulsatile control strategy.

Importantly, for both pump types, the proposed ESV control algorithm with pulsatility resulted in lower mean pump speeds while maintaining the desired mean PF, ejection fraction, and mean AoP, as summarized in Tables 4 and 5 (FIGS. 9 and 10 respectively). Furthermore, no suction events were observed under any simulated condition, indicating robust ventricular unloading.

For the axial LVAD, the introduction of LVV measurement noise under both normal and elevated PVR conditions, at rest and during exercise, led to increases in pulsatility frequency, mean pump speed, and mean pump flow. Nevertheless, cardiac output remained within the desired physiological range without suction events, and mean aortic pressure was effectively preserved, demonstrating the noise robustness of the proposed control strategy (Table 4 shown in FIG. 9). In contrast, for the centrifugal LVAD, mean pump speed, mean pump flow, and mean aortic pressure remained largely unaffected by the addition of measurement noise across all conditions, except during exercise with 8×PVR, where pulsatility frequency increased from 2 to 10 cycles per minute. However, this increase did not compromise the ability of the proposed ESV control algorithm to maintain sufficient cardiac output and prevent LV suction (Table 5 shown in FIG. 10).

When LVV measurement noise was added under normal or elevated PVR at rest or exercise, the performance of both LVADs was evaluated. For the axial LVAD, the pulsatility frequency, mean pump speed, and mean pump flow all increased. Nevertheless, the cardiac output remained within the desired physiological range without suction events, and mean aortic pressure was effectively preserved, demonstrating the noise resilience of the pump-independent control algorithm (Table 4 shown in FIG. 9). For the centrifugal LVAD, the addition of LVV measurement noise resulted in the mean pump speed, pump flow, and aortic pressure remaining almost constant across all conditions, except during exercise with 8×PVR, where the pulsatility frequency increased from 2 to 10 cycles per minute. However, this phenomenon did not affect the proposed ESV control algorithm's ability to produce sufficient cardiac output and effectively prevent LV suction (Table 5 shown in FIG. 10).

### Performance of the Pump-Independent Control Algorithm at Rest With Pulsatility With Different ESV_{Hr} Values

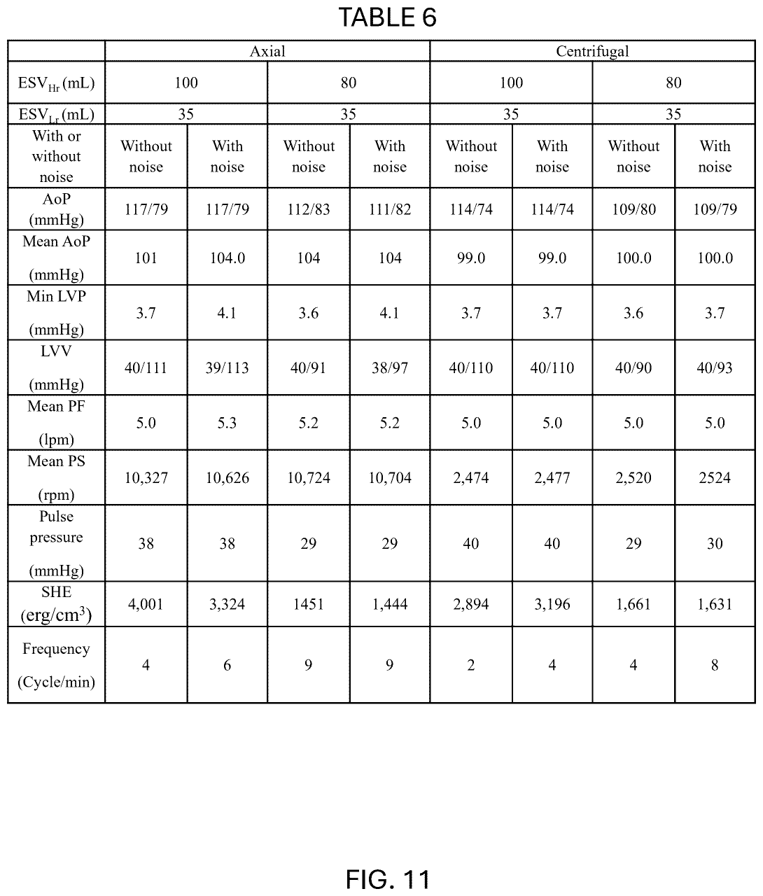

The impact of modulating the ESV high setpoint (ESV_{Hr}) on pulsatility characteristics is summarized in Table 6 (FIG. 11). Reducing ESVHr from 90 mL to 80 mL increased pulsatility frequency from 4 to 9 cycles/min (axial LVAD) and 2 to 4 cycles/min (centrifugal LVAD) under noise-free conditions. However, this adjustment reduced pulse pressure from 38 mmHg to 29 mmHg (axial) and 40 mmHg to 29-30 mmHg (centrifugal). Similar trends persisted under 6% measurement noise, with axial and centrifugal LVADs maintaining pulse pressures of 29-30 mmHg at frequencies of 9 and 8 cycles/min, respectively. Notably, mean aortic pressure (101-104 mmHg), and pump flow (5.0-5.3 L/min), demonstrating that pulsatility frequency modulation does not compromise hemodynamic stability. Table 6 (FIG. 11) shows the effect of ESV_{Hr} modulation (90 vs. 80 mL) on pulsatility characteristics in axial and centrifugal LVADs under varying noise conditions.

### Performance of the Pump-Independent Control Algorithm Under the Condition of 8-Time PVR

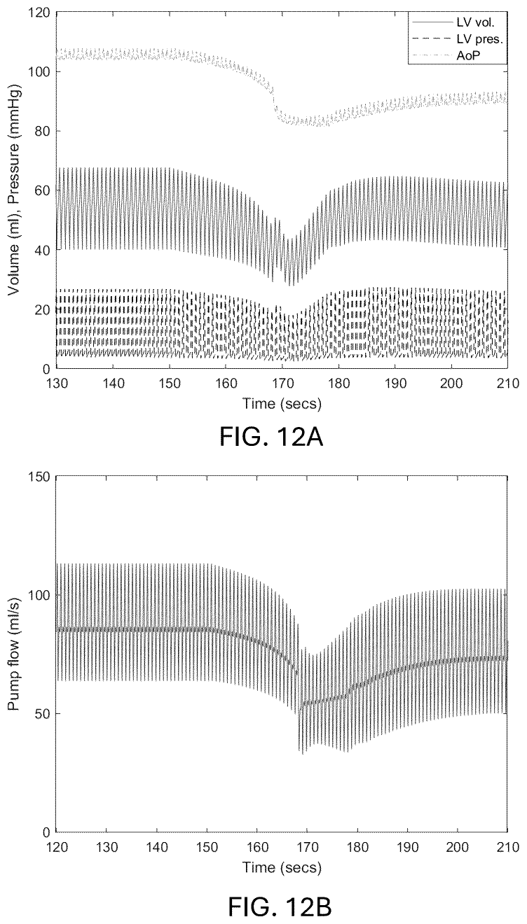

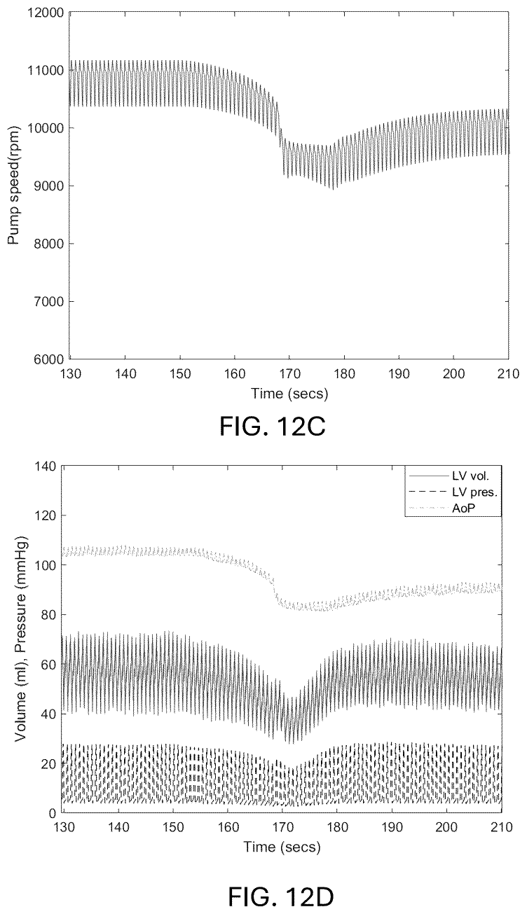

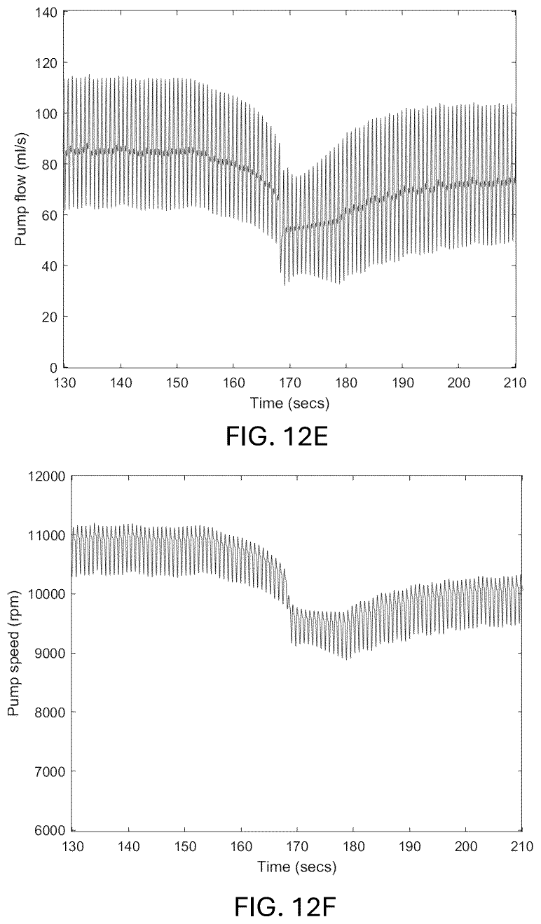

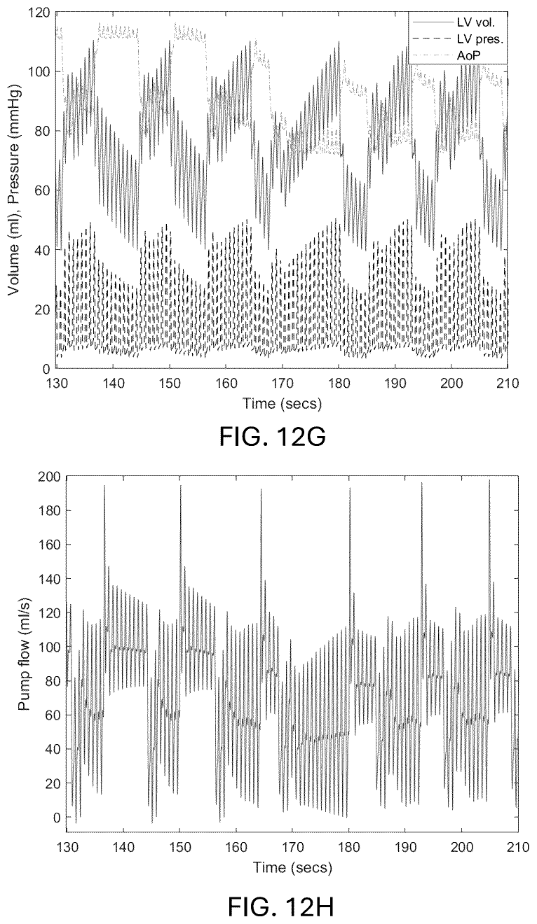

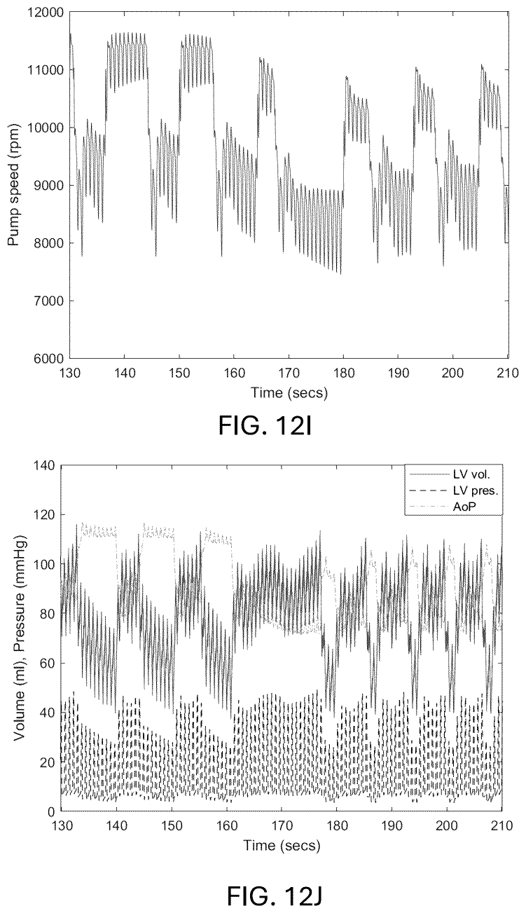

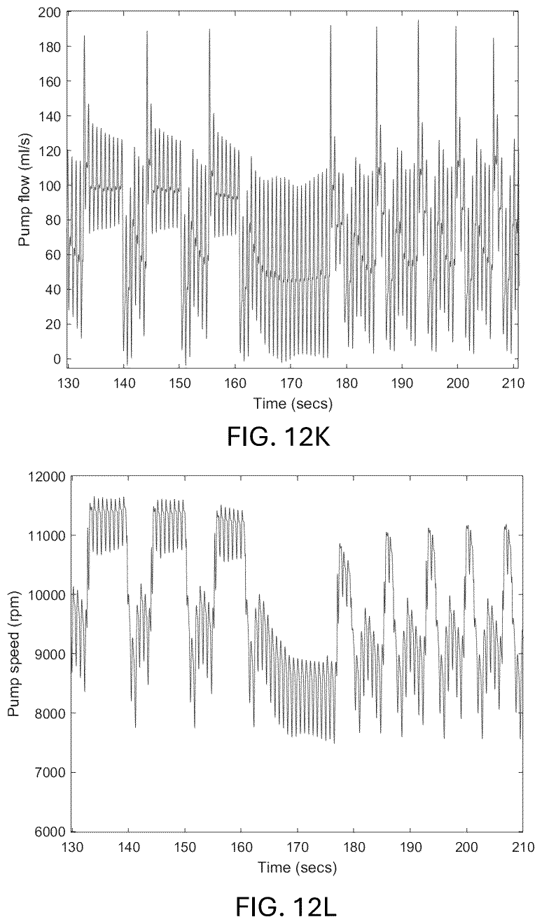

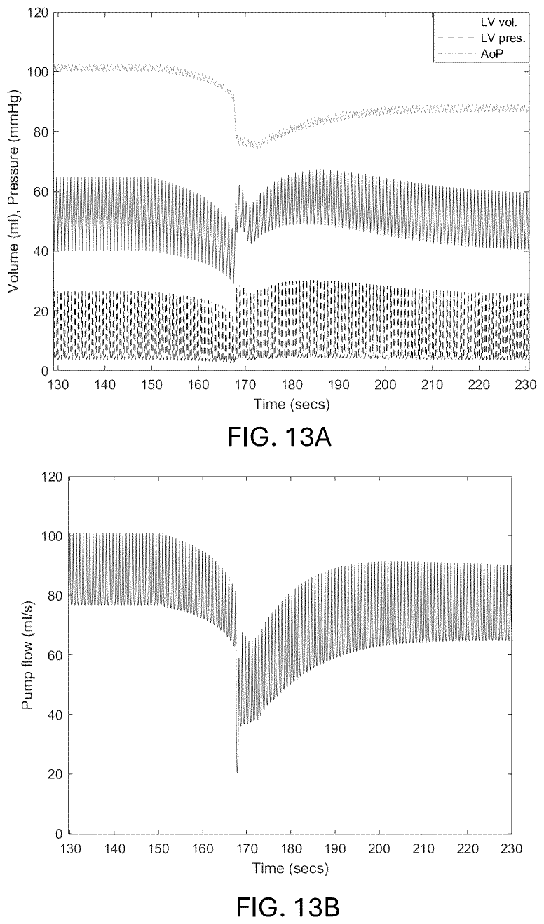

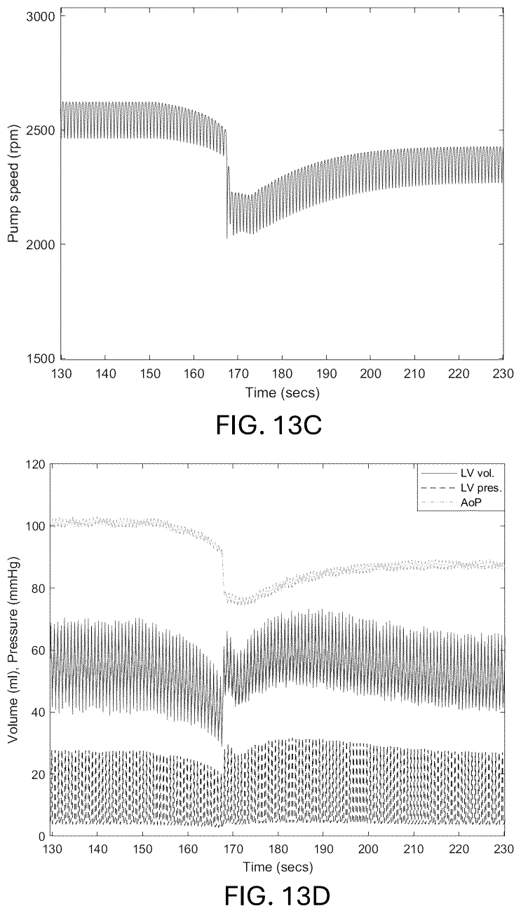

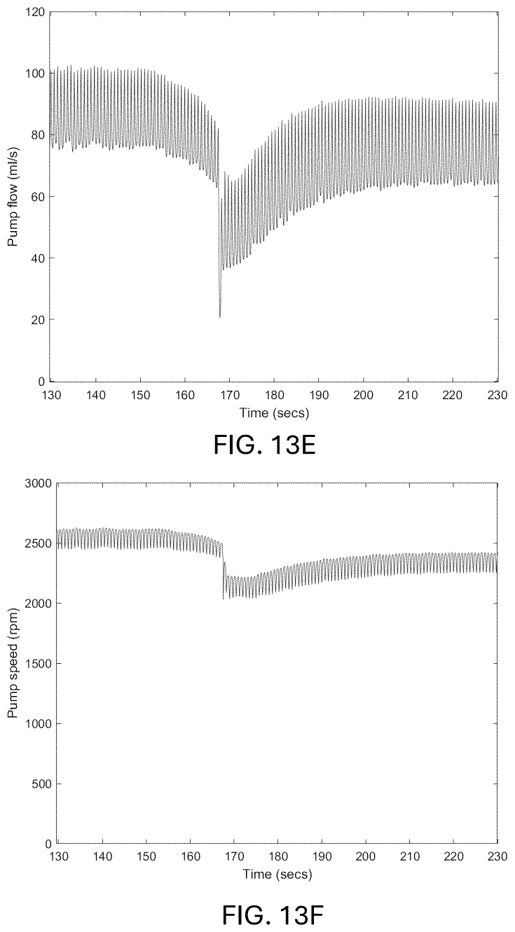

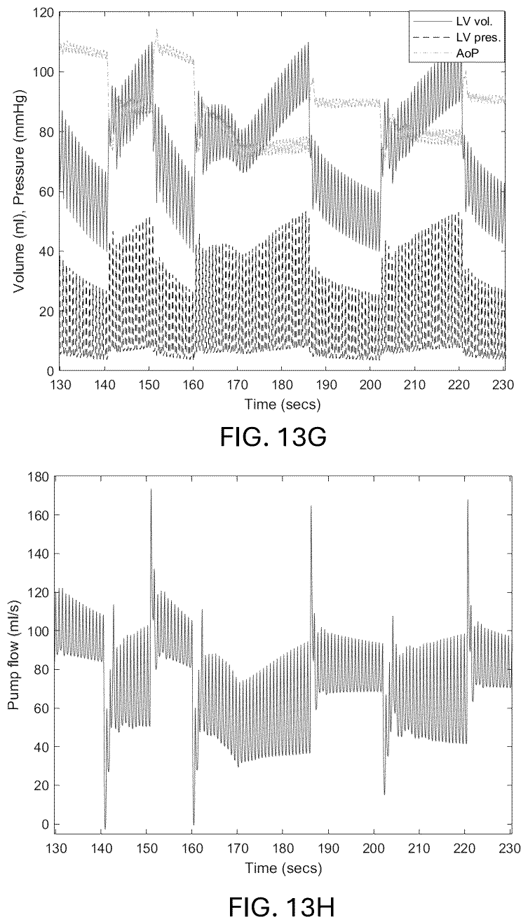

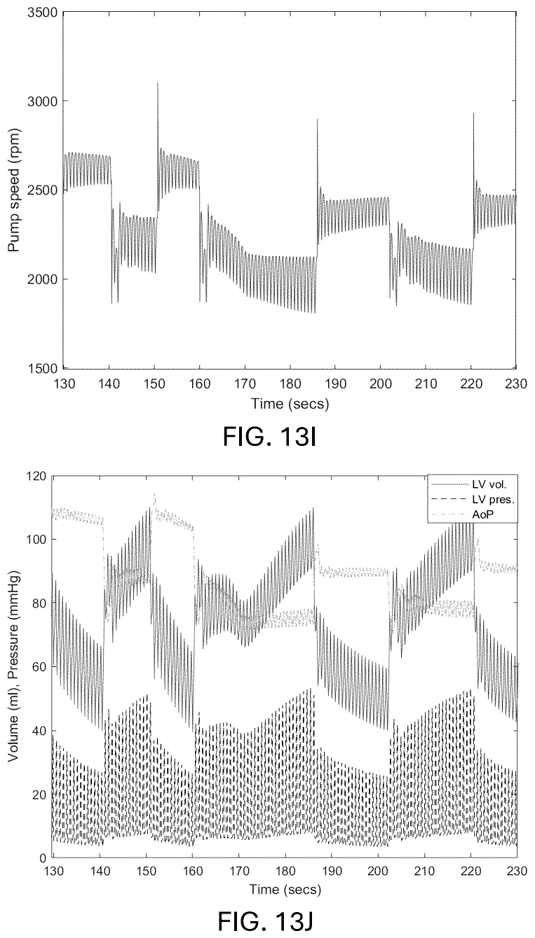

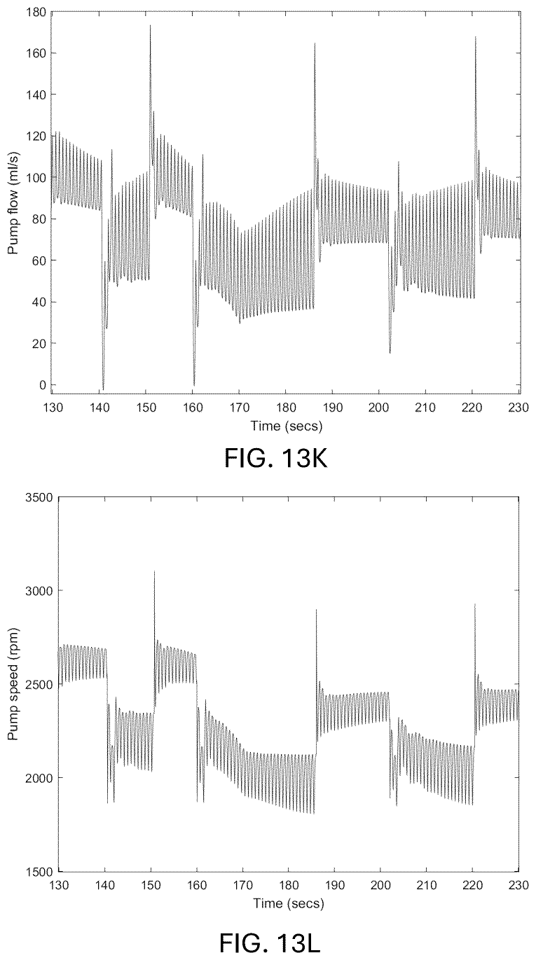

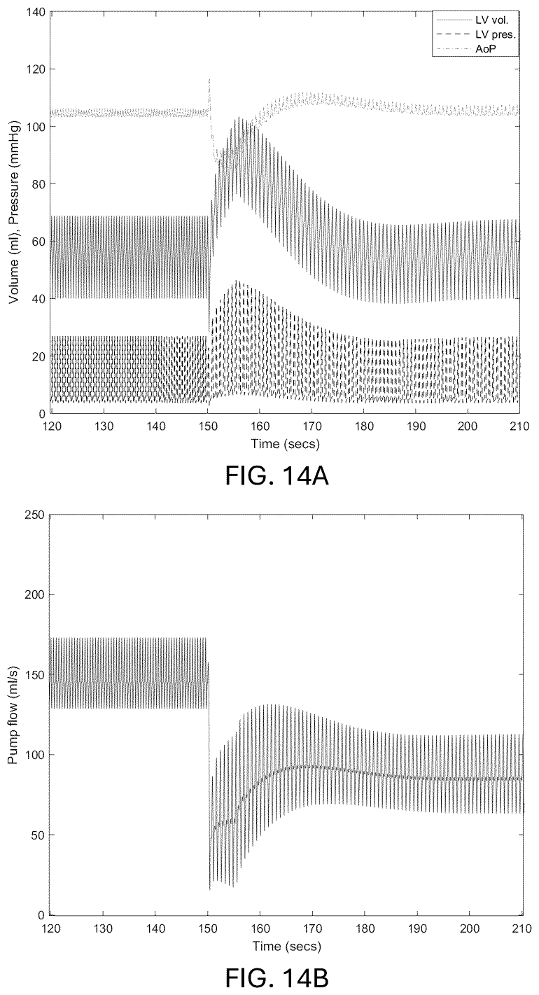

FIGS. 12A-12L and 13A-13L show the in-silico results of the proposed pump-independent control algorithm during a gradual increase in PVR to eight times the baseline value over 20 seconds, beginning at t=150 s, for both the axial and centrifugal LVADs, with and without added LVV measurement noise. With pulsatility mode enabled, the transition from normal PVR to 8×PVR was smooth, without suction events. The safe mode was not triggered, as the LVV consistently remained above 30 mL and the LVP remained well above 3 mmHg. In contrast, without pulsatility mode, both LVADs exhibited a drop in LVV below 30 mL during the PVR increase, triggering activation of the safe mode. The safe mode successfully operated the LVADs at a current for 5 seconds, allowing LVV to recover above 30 mL, maintaining LVP above 3 mmHg, and preventing suction events.

Across all cases, the transition from normal to 8×PVR resulted in reductions in mean aortic pressure (AoP) and pump flow (PF), regardless of the presence of LVV measurement noise. These reductions indicated that physiological perfusion under the elevated PVR condition was clearly lower than under normal rest and exercise conditions (Tables 4 and 5 shown in FIGS. 9 and 10 respectively). Regarding pulsatility frequency, it increased in the axial flow LVAD, both with and without added LVV measurement noise. In the centrifugal flow LVAD, the pulsatility frequency slightly decreased at rest but increased dramatically during exercise, reaching 10 cycles per minute.

FIGS. 12A-12L show in-silico results of the pump-independent control algorithm at rest under the condition of 8-time PVR starting at t=150 s for the axial LVAD. FIGS. 12A-12C show in-silico results without pulsatility and without LVV measurement noise. FIGS. 12D-12F show in-silico results without pulsatility and with LVV measurement noise. FIGS. 12G-12I show in-silico results with pulsatility and without LVV measurement noise. FIGS. 12J-12L show in-silico results with pulsatility and with LVV measurement noise. FIGS. 13A-13L show in-silico results of the pump-independent control algorithm at rest under the condition of 8-time PVR starting at t=150 s for the centrifugal LVAD. FIGS. 13A-13C show in-silico results without pulsatility and without LVV measurement noise. FIGS. 13D-13F show in-silico results without pulsatility and with LVV measurement noise. FIGS. 13G-13I show in-silico results with pulsatility and without LVV measurement noise. FIGS. 13J-13L show in-silico results with pulsatility and with LVV measurement noise.

### Performance of the Pump-Independent Control Algorithm During the Transient Change

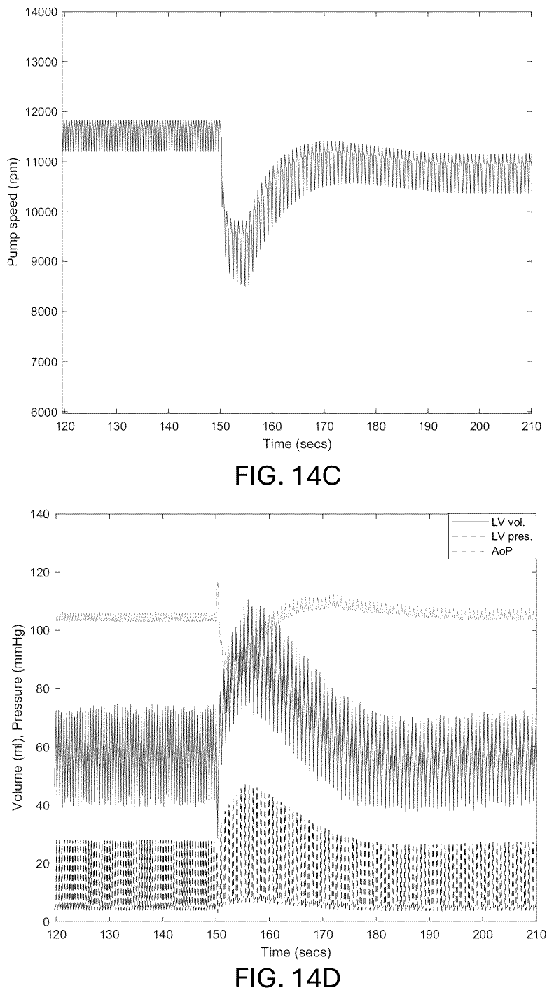

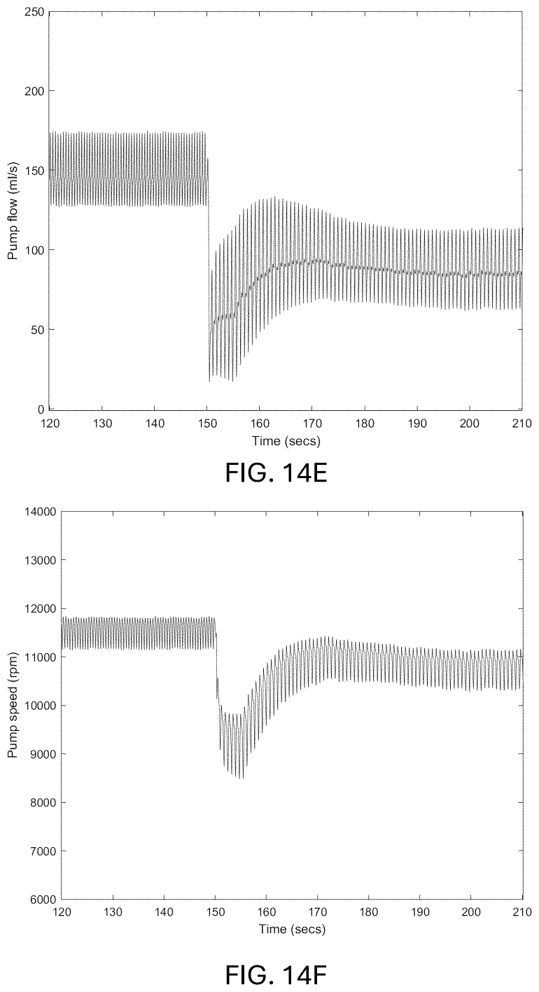

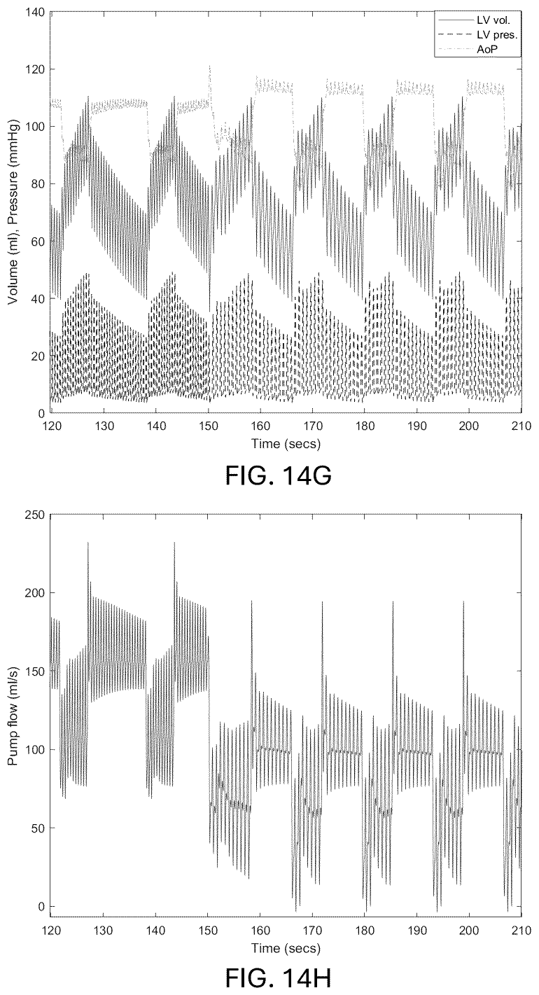

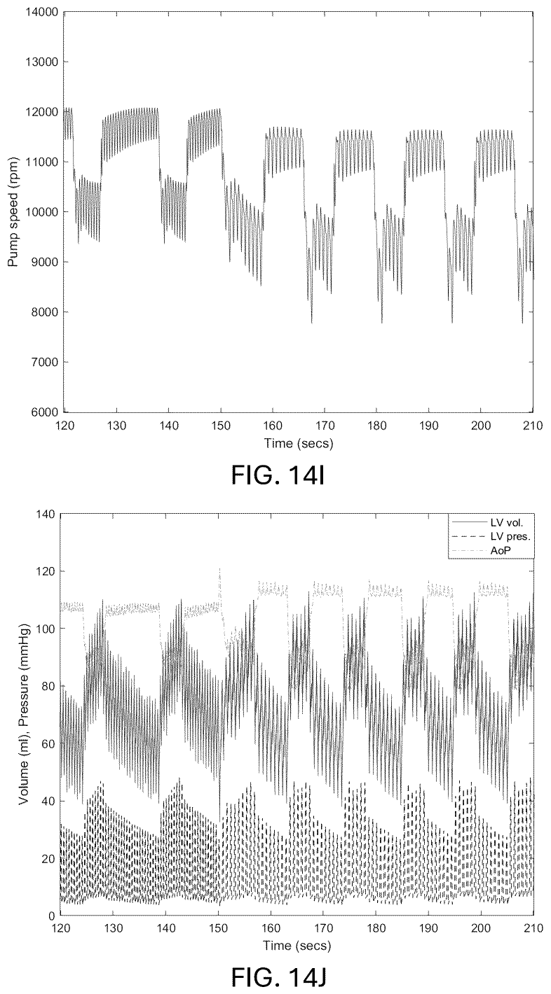

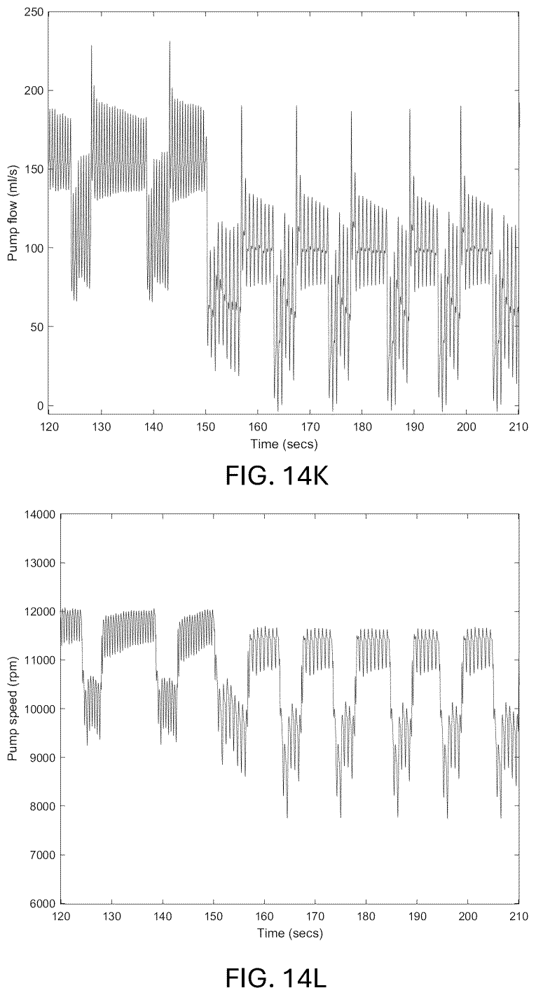

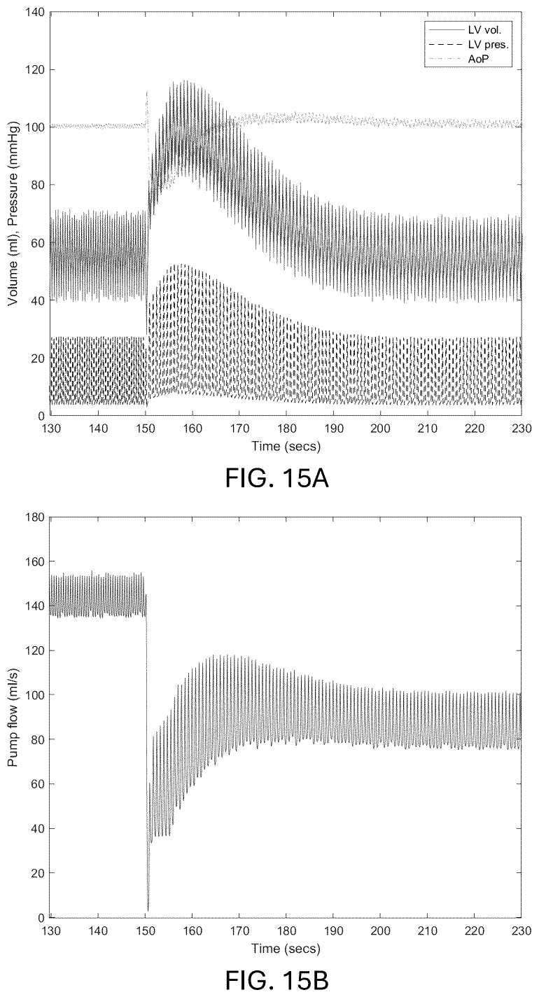

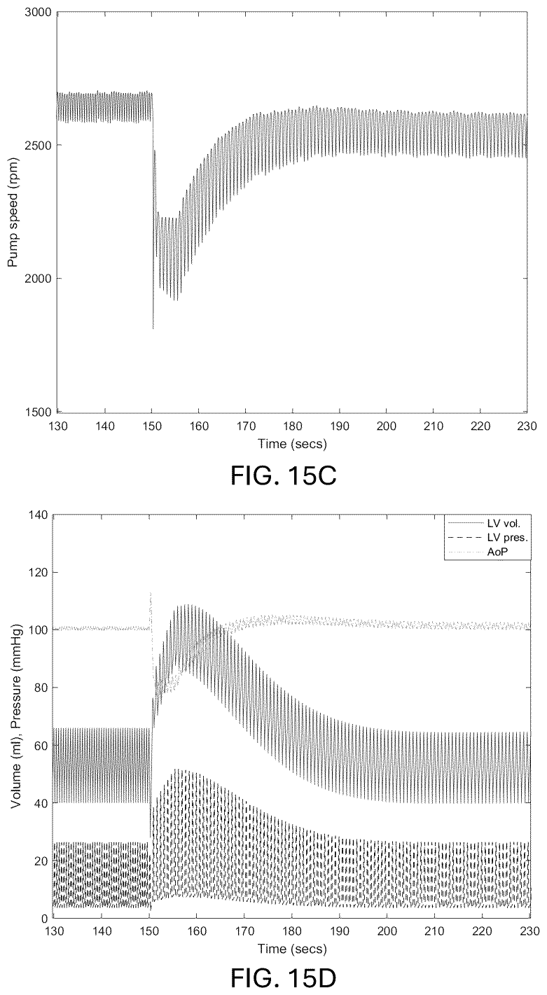

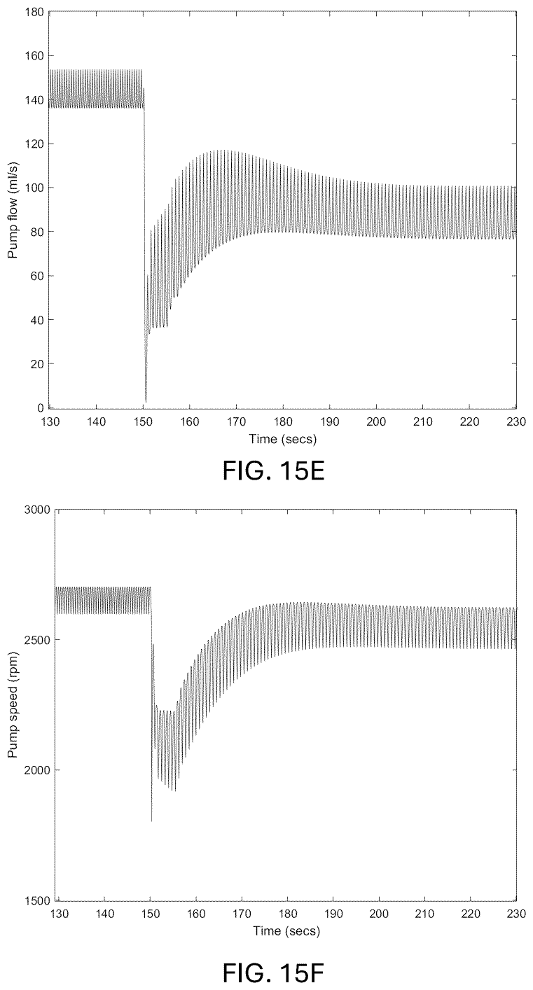

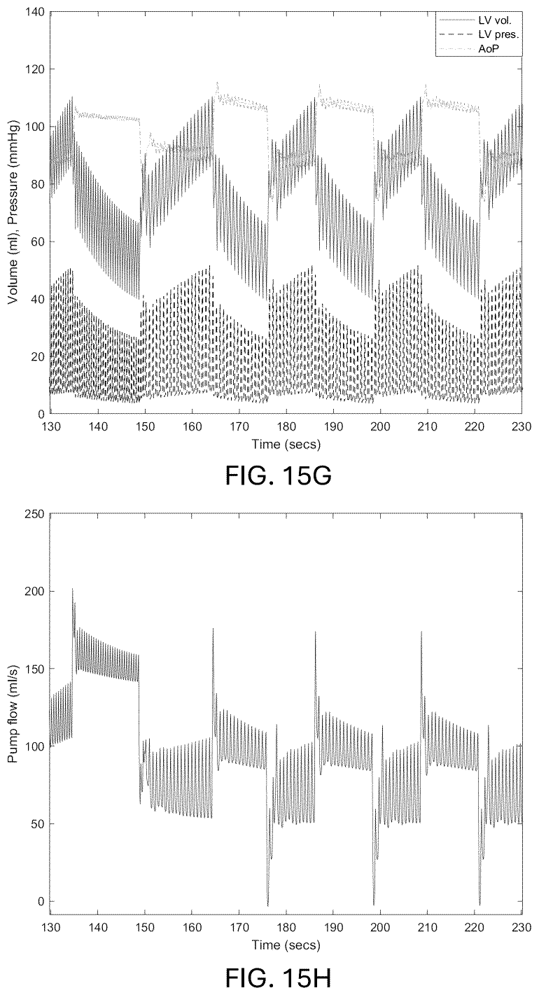

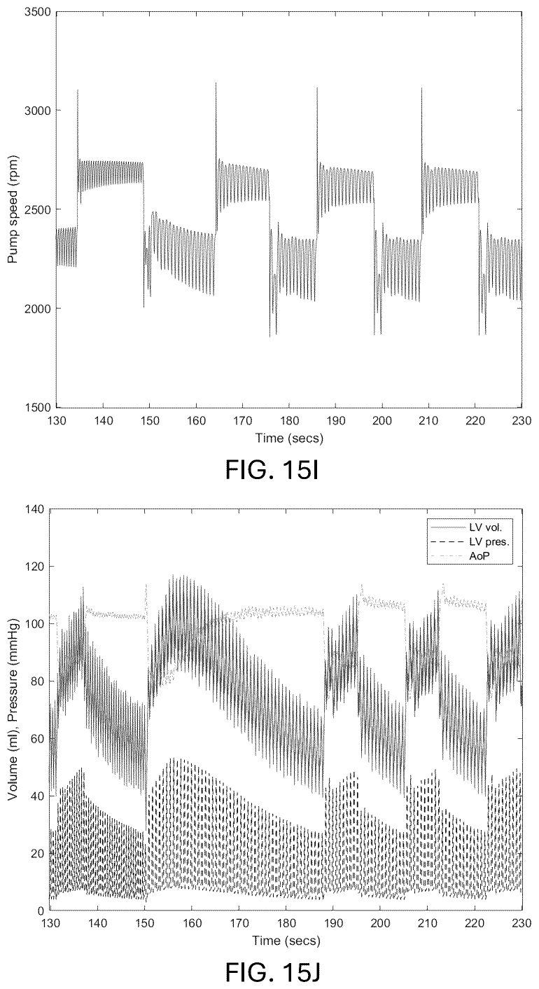

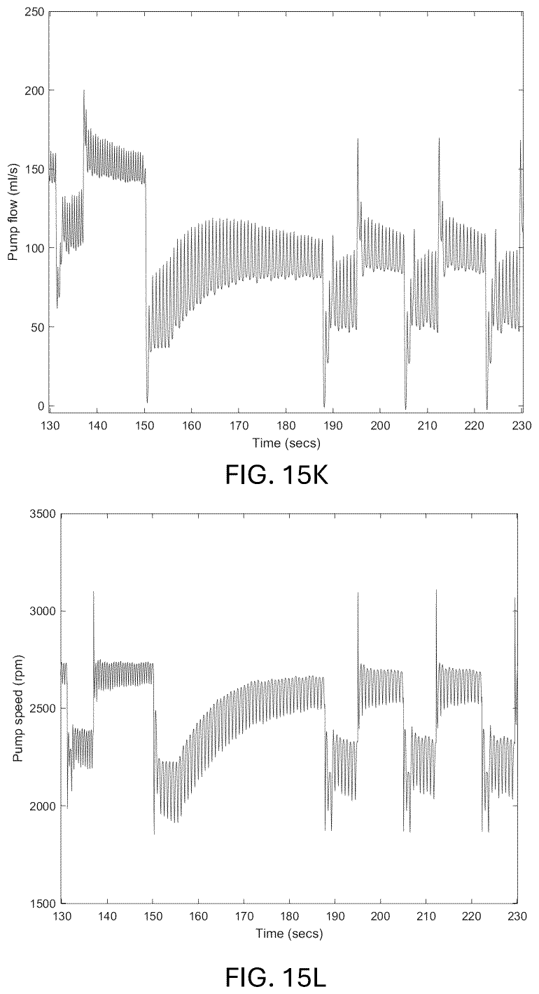

FIGS. 14A-14L and 15A-15L show the in-silico results of the proposed pump-independent control algorithm during a sudden transition from exercise to rest at t=150 s for both axial and centrifugal LVADs, under conditions with and without LVV measurement noise. The controller effectively augmented physiological perfusion during exercise and appropriately reduced it during rest, maintaining flow levels markedly higher than those observed without LVAD support (Tables 4 and 5 in FIGS. 9 and 10 respectively).

Similar to the transition from normal to 8-fold PVR without pulsatility, both LVADs exhibited a transient drop in LVV below 30 mL during the exercise-to-rest shift, prompting activation of the safe mode. The safe mode successfully maintained pump operation at a fixed current for 5 seconds, allowing LVV to recover above 30 mL and preventing suction events across all cases. By contrast, with the pulsatility mode enabled, the transition remained smooth without triggering the safe mode, as LVV consistently stayed above the suction threshold. No suction events were observed under any condition. It is important to note that results for the transition from rest to exercise were not shown, as the exercise-to-rest transition presents a greater risk for LV suction.

FIGS. 14A-14L show in-silico results of the pump-independent control algorithm during the transient change from exercise to rest at t=150 s for the axial LVAD. FIGS. 14A-14C show in-silico results without pulsatility and without LVV measurement noise. FIGS. 14D-14F show in-silico results without pulsatility and with LVV measurement noise. FIGS. 14G-14I show in-silico results with pulsatility and without LVV measurement noise. FIGS. 14J-14L show in-silico results with pulsatility and with LVV measurement noise. FIGS. 15A-15L show in-silico results of the pump-independent control algorithm during the transient change from exercise to rest at t=150 s for the centrifugal LVAD. FIGS. 15A-15C show in-silico results without pulsatility and without LVV measurement noise. FIGS. 15D-15F show in-silico results without pulsatility and with LVV measurement noise. FIGS. 15G-15I show in-silico results with pulsatility and without LVV measurement noise. FIGS. 15J-15L show in-silico results with pulsatility and with LVV measurement noise.

### Discussion

The simulation results provide significant insights into the cardiovascular system's behavior in heart failure patients without LVAD support. The baseline analysis revealed distinct physiological adaptations between rest and exercise states. At rest, the cardiac output was reduced compared to healthy physiology, while the LVV ranged within expected limits for heart failure conditions. During exercise, CO increased to meet higher metabolic demands, and LVV showed corresponding changes, reflecting the system's compensatory response. These findings highlight the cardiovascular limitations in heart failure patients while establishing a baseline for evaluating the impact of LVAD intervention and advanced control strategies.

Under normal rest and exercise conditions, the developed LVAD control algorithm demonstrated its capability to adaptively support physiological demands. At rest, the LVAD maintained hemodynamic stability, as indicated by the mean AoP, consistent LVP, and appropriate LVV ranges. During exercise, the system effectively increased pump flow and pump speed, reflecting the higher CO requirements. These findings underscore the control algorithm's ability to enhance cardiac function while preserving left ventricular stability across varying conditions.

The in-silico results validated the hypothesis that the developed pump-independent control algorithm can regulate pump speed to meet physiological requirements while effectively preventing LV suction events across a variety of conditions. This was achieved through the precise measurement of LVV using a newly developed, non-blood-contact sensor system. The ESV control algorithm maintained stable operation even under extreme conditions, such as an abrupt eightfold increase in PVR—a severe and non-physiological scenario that may occur during mild coughing or a Valsalva maneuver. Similarly, the system successfully managed transient shifts between rest and exercise, another challenging and highly dynamic situation. These simulations demonstrate that the ESV pump-independent control algorithm can maintain sufficient cardiac output while providing robust protection against suction events.

The inverse relationship between pulsatility frequency and pulse pressure observed in this study highlights a critical trade-off in LVAD control design. While reducing ESV_{Hr} from 90 mL to 80 mL doubled pulsatility frequency (4→9 cycles/min for axial, 2→4 cycles/min for centrifugal), it concurrently lowered pulse pressure below the clinically critical threshold of 35 mmHg, increasing risks of vascular complications such as gastrointestinal bleeding [72]. This suggests that optimizing pulsatility requires balancing frequency and amplitude to align with physiological norms (60-100 cycles/min and >35 mmHg, respectively). Future algorithms could incorporate adaptive ESV_{Hr} modulation based on real-time metabolic demand, enabling context-aware adjustments. Despite this limitation, stable aortic pressures and ventricular volumes across all ESV_{Hr} configurations underscore the robustness of the pump-independent control algorithm in maintaining perfusion while exploring pulsatility parameters.

At the heart of the control system is the accurate estimation of ESV. Resonantly coupled sensors, as described in [74], enabled this prediction by exploiting the highly sensitive relationship between spatial separation and the transmission coefficient, effectively modeled through polynomial regression. Using this approach, the distance between the sensors—and consequently the size of the LV chamber, including ESV—could be estimated with 95% accuracy and no significant drift observed. To further assess the robustness of the control system, uniformly distributed noise (6%) was introduced into the LVV measurements. The results showed that the presence of measurement noise did not compromise the performance of the ESV control strategy, eliminating the need for implantable pressure or flow sensors and enhancing the reliability and safety of the rotary blood pump (RBP) system.

The control algorithm is pump-independent because it regulates pump speed based solely on physiological parameters, specifically ESV, rather than relying on embedded flow or pressure sensors traditionally tied to specific pump designs. By focusing on LVV dynamics, the algorithm can be universally applied to both axial and centrifugal pumps, enhancing adaptability across devices. This independence not only enhances the flexibility and adaptability of the control system but also improves the overall safety and reliability of mechanical circulatory support, eliminating the risks and complications associated with implantable pressure or flow sensors.

### Maintaining a Physiologically Relevant Pulse Pressure is Essential for

Reducing complications like gastrointestinal bleeding and preserving vascular endothelial function [87]. The ESV control algorithm successfully achieved a pulse pressure of 38 and 40 mmHg, for axial and centrifugal LVADs, respectively, at rest with normal PVR, exceeding the minimum target of 35 mmHg established in the literature [68, 87]. This result supports the preservation of vascular endothelial function and reduces the risk of von Willebrand factor degradation [88]. Additionally, the control algorithm maintained a pulsatility rate of approximately 4 and 2 cycles per minute, for axial and centrifugal LVADs, respectively, demonstrating its capability to mimic native cardiac behavior.

Beyond restoring pulse pressure, the ESV control algorithm with pulsatility significantly reduced the mean pump speed compared to conventional continuous-flow control strategies. This reduction in pump speed offers several advantages: it decreases pump-generated noise, which is a common patient complaint, and lowers the electrical current consumption, thereby improving the overall battery life and extending the duration of portable use for patients. Importantly, despite the lower pump speeds, the algorithm-maintained CO and mean AoP within the physiological range, ensuring adequate systemic perfusion, as demonstrated in prior studies of adaptive LVAD control strategies [77]. Furthermore, by operating at reduced speeds, the LVADs generated lower shear stress on blood cells, which can substantially decrease the risk of blood damage and hemolysis [89], contributing to better long-term biocompatibility and patient outcomes.

The safe mode in the LVAD control algorithm demonstrated a crucial ability to address scenarios of reduced venous return by dynamically adjusting pump speed to maintain hemodynamic stability. As highlighted in the results, the activation of the safe mode effectively prevented left ventricular collapse and suction events, which are major concerns in continuous flow LVADs that lack load-responsive mechanisms. This feature ensured continuous and stable cardiac output even when venous return was temporarily reduced, such as during activity changes or increased PVR.

The results showed that during periods of low venous return, the safe mode limited excessive ventricular unloading by decreasing pump speed in real-time. This adaptive behavior preserved adequate left ventricular filling, avoided over-drainage of the ventricle, and maintained flow rates within physiologically acceptable ranges. These results highlight the safe mode's critical role in supporting natural cardiac loading conditions, reducing the likelihood of complications associated with aggressive or unregulated unloading.

The feasibility of the ESV control algorithm has been demonstrated but has several limitations. The lumped parameter model (LPM) used simplifies physiological processes, assuming blood behaves as a Newtonian fluid, heart valves function perfectly without regurgitation or pressure drops, and omitting gravitational and inertial effects. These assumptions limit the model's ability to capture complex phenomena such as baroreceptor responses, tissue remodeling, and neurohumoral mechanisms. Additionally, the model cannot fully replicate long-term physiological responses, or the variability seen across individual patients. The pump-independent control algorithm can be further improved by adding an objective to ensure periodic opening of the aortic valve to mitigate valve fusion. Furthermore, while the algorithm restored arterial pulse pressure, its subphysiological pulsatility frequency may limit hemodynamic and endothelial benefits compared to natural cardiac pulsation, necessitating future work to optimize pulsatility rates through. Despite these constraints, valuable insights are obtained into the control algorithm's potential, laying the groundwork for future validation through mock circulatory flow loops and large animal studies to assess safety, efficacy, and adaptability under more realistic conditions.

The feasibility of a pump-independent, end-systolic volume (ESV)-based control algorithm for axial and centrifugal left ventricular assist devices (LVADs) has been demonstrated, achieving robust physiological perfusion and ventricular unloading across diverse clinical scenarios. By maintaining a fixed ESV of 40 mL, the pump-independent control algorithm ensured sufficient cardiac output (5.0-8.5 L/min) and mean aortic pressure (83-104 mmHg) while preventing suction events, even during abrupt transitions in pulmonary vascular resistance (PVR) or activity levels. The integration of pulsatility restored physiologically relevant pulse pressures (23-40 mmHg) and improved ejection fraction (64%) compared to non-pulsatile operation, though pulsatility frequency remained subphysiological (2-4 cycles/min) at rest. The pump-independent control algorithm exhibited resilience to 6% ventricular volume measurement noise and adaptability to both pump types, with centrifugal LVADs showing superior noise tolerance. The implementation of a safety mode effectively mitigated suction risks during transient hemodynamic shifts, underscoring its clinical utility. While the in-silico model simplified complex physiological interactions, these results validate the algorithm's potential to address key limitations of current LVAD therapies, such as vascular stiffening and hemolysis, through reduced pump speeds and shear stress. Future work should prioritize in vivo validation, optimization of pulsatility synchronization with native autonomic rhythms, and incorporation of patient-specific variability to advance toward personalized, next-generation mechanical circulatory support.

### REFERENCES

[1] Virani SS, Alonso A, Aparicio HJ, et al. Heart disease and stroke statistics—2021 update: a report from the American Heart Association. Circulation. 2021; 143: e254-743. Doi: 10.1161/CIR.0000000000000950.
[2] Shahim B, Kapelios CJ, Savarese G, et al. Global public health burden of heart failure: an updated review. Card Fail Rev 2023, 9: e11. Doi: 10.15420/cfr.2023.05.
[3] E. J. Benjamin et al., “Heart Disease and Stroke Statistics—2017 Update: A Report From the American Heart Association,” Circulation, vol. 135, no. 10, pp. E646, Mar. 2017.
[4] M. A. Simaan, A. Ferreira, S. Chen, J. F. Antaki, and D. G. Galati, “A dynamical state space representation and performance analysis of a feedback-controlled rotary left ventricular assist device,” IEEE Trans Control Syst Technol., vol. 17, no. 1, pp. 15-28, Jan. 2009.
[5] D. O. Taylor et al., “Registry of the international society for heart and lung transplantation: Twenty-fourth offificial adult heart transplant report—2007,” J. Heart Lung Transp., vol. 26, no. 8, pp. 769-781, Aug. 2007.
[6] Rogers, J.G.; Pagani, F.D.; Tatooles, A.J.; Bhat, G.; Slaughter, M.S.; Birks, E.J. et al., Intrapericardial Left Ventricular Assist Device for Advanced Heart Failure. N Engl J Med 2017, 376 (5), 451-460. https://doi.org/10.1056/NEJMoa1602954.
[7] Slaughter, M.S.; Rogers, J.G.; Milano, C.A.; Russell, S.D.; Conte, J.V.; Feldman, D. et al., Advanced Heart Failure Treated with Continuous-Flow Left Ventricular Assist Device. N Engl J Med 2009, 361 (23), 2241-2251. https://doi.org/10.1056/NEJMoa0909938.
[8] Schumer, E.M.; Black, M.C.; Monreal, G.; Slaughter, M.S. Left Ventricular Assist Devices: Current Controversies and Future Directions. Eur Heart J 2016, 37 (46), 3434-3439. https://doi.org/10.1093/eurheartj/ehv590.
[9] Smith, P.A.; Wang, Y.; Bieritz, S.A.; Sampaio, L.C.; Cohn, W.E.; Metcalfe, R.W. et al., Design Method Using Statistical Models for Miniature Left Ventricular Assist Device Hydraulics. Ann Biomed Eng 2019, 47 (1), 126-137. https://doi.org/10.1007/s10439-018-02140-w.
[10] R. F. Salamonsen, D. G. Mason, and P. J. Ayre, “Response of rotary blood pumps to changes in preload and afterload at a fixed speed setting are unphysiological when compared with the natural heart,” Artif Organs, vol. 35, no. 3, pp. E47-E53, Mar. 2011.
[11] G. A. Giridharan, S. C. Koenig, and M. S. Slaughter, “Do axial-flow LVADs unload better than centrifugal-flow LVADs?,” ASAIO J., vol. 60, no. 2, pp. 137-139, Mar./Apr. 2014.
[12] Harjola VP, Mullens W, Banaszewski M, et al., Organ dysfunction, injury and failure in acute heart failure: from pathophysiology to diagnosis and management. A review on behalf of the acute heart failure committee of the heart failure association (HFA) of the European society of cardiology (ESC). European J Heart Fail. 2017, 19 (7), 821-836.
[13] A. Sen et al., “Mechanical circulatory assist devices: a primer for critical care and emergency physicians,” Crit Care., vol. 20, no. 1, pp. 153, Jun. 2016.
[14] Son J, Du D, Du Y. Modelling and control of a failing heart managed by a left ventricular assist device. Biocybernetics and Biomedical Engineering, 40, 2020, 559-573.
[15] Zeile C, Rauwolf T, Schmeisser A, et al., An intra-cycle optimal control framework for ventricular assist devices based on atrioventricular plane displacement modeling. Ann Biomed Eng 2021, 49 (12), 3508-3523.
[16] Wang Y, Koenig SC, Slaughter MS, Giridharan GA*. Suction prevention and physiologic control of continuous flow left ventricular assist devices using intrinsic pump parameters. ASAIO Journal, 61 (2): 170-177, 2015.
[17] Ketelhut M, Stemmler S, Gesenhues J, et al., Iterative learning control of ventricular assist devices with variable cycle durations. Control Engineering Practice, 83, 2019, 33-44.
[18] Fetanat M, Stevens, Hayward C, et al., A physiological control system for an implantable heart pump that accommodates for interpatient and intrapatient vaviations. IEEE TBME, 67 (4), 2020, 1167-1175.
[19] Liu X, Qu H, Meng L, et al., A physiological control method based on SMC and GAPSO for artificial heart pumps to maintain pulsatility and avoid regurgitation and suction. J Medical Biological Engineering 43, 2023: 42-51.
[20] Fetanat M, Stevens M, Hayward C, et al., A sensorless control system for an implantable heart pump using a real-time deep convolutional neural network. IEEE Trans Biomed Eng. 68 (10), 2021, 3029-3038.
[21] Li T, Cui W, Xie N, et al., Intelligent and strong robust CVS-LVAD control based soft-actor-critic algorithm. Artificial Intelligence in Medicine. 128, 2022: 102308.
[22] Magkoutas K, Arm P, Meboldt M, et al., Physiologic data-driven iterative learning control for left ventricular assist devices. Front. Cardiovasc. Med. 9:922387. doi: 10.3389/fcvm.2022.922387.
[23] Bakouri M, Alassaf A, Alshareef K, et al., A feasible method to control left ventricular assist devices for heart failure patients: a numerical study. Mathematics 2022, 10, 2251. doi.org/10.3390/math10132251
[24] Bakouri M, Alassaf A, Alshareef K, et al., In-silico evaluation of a physiological controller for a rotary blood pump based on a sensorless estimator. Appl. Sci. 2022, 12, 11537. doi.org/10.3390/app122211537.
[25] Melo TR, Neto, JSR, IA, Cestari et al., Feedback controller for restoring the basal hemodynamic condition with a rotary blood pump used as left ventricular assist device. BSPC, 62, 2020, 102136.
[26] Magkoutas K, Rossato LN, Heim M, et al., Genetic algorithm-based optimization framework for control parameters of ventricular assist devices. BSPC, 85, 2023, 104788.
[27] Wu EL, Stevens MC, Nestler F, et al., A starling-like total work controller for rotary blood pumps: an in vitro evaluation. Artif. Organs, vol. 44, no. 3, pp. E40-E53, Mar. 2020.
[28] Stephens AF, Busch A, Salamonsen RF, et al., Rotary ventricular assist device control with a fiber bragg grating pressure sensor. TBME, 29 (3), 2021, 1009-1018.
[29] Borchers P, Walter M, Leonhardt S, et al., Flow profile generation for a left ventricular assist device using iterative learning control, Current Directions BME, 7 (2), 2021, 279-282.
[30] Li T, Li H, Cui W, et al., Design and intelligent control of mock circulation system to reproduce patient-specific physiological indexes. BSPC, 78, 103987, 2022.
[31] Li T, Cui W, Liu X, et al., A physiological control system for pulsatile ventricular assist device using an energy-efficient deep reinforcement learning method, TIM, 72, 3517910, 2023.
[32] Schaefer A-K, Schima M, Wittmann F, Wiedemann D, Moscato F, Kudlik D, Stadler R, Zimpfer D and Schima H (2022) A Sensorless Modular Multiobjective Control Algorithm for Left Ventricular Assist Devices: A Clinical Pilot Study. Front. Cardiovasc. Med. 9:888269. doi: 10.3389/fcvm.2022.888269
[33] Cysyk J, Jhun CS, Newswanger R, et al., In vivo evaluation of a physiologic control system for rotary blood pumps based on the left ventricular pressure-volume loop, ASAIO J, 68 (6), 2022, 791-799.
[34] Habigt MA, Hein M, Gesenhues J, et al., In vivo evaluation of a novel control algorithm for left ventricular assist devices based upon ventricular stroke work, ASAIO J, 69 (1), 2023, 86-95.
[35] Liang, L.; Meki, M.; Wang, W.; Sethu, P.; El-Baz, A.; Giridharan, G. A.; Wang, Y. A Suction Index Based Control System for Rotary Blood Pumps. Biomedical Signal Processing and Control 2020, 62, 102057. https://doi.org/10.1016/j.bspc.2020.102057.
[36] S. Bozkurt, S. van Tuijl, S. Schampaert, F.N. van de Vosse, M.C.M. Rutten, Arterial pulsatility improvement in a feedback-controlled continuous flow left ventricular assist device: an ex-vivo experimental study, Med. Eng. Phys 36 (10) (2014) 1288-1295.
[37] J.P. Paul, M.C. Stevens, N. Bartnikowski, J.F. Fraser, S.D. Gregory, G. Tansley, Evaluation of physiologic control systems for rotary left ventricular assist devices: an in-vitro study, Ann. Biomed. Eng. 44 (8) (2016) 2377-2387.
[38] Tan Z, Huo M, Qin K, et al., A sensorless, physiologic feedback control strategy to increase vascular pulsatility for rotary blood pumps, Biomedical Signal Processing and Control 2023, 83, 104640.
[39] Wang Y, Koenig SC, Wu ZJ, Slaughter MS, Giridharan GA*. Sensor-based physiologic control strategy for biventricular support with rotary blood pumps. ASAIO Journal, 64(3):338-350, 2018.
[40] Wang Y, Koenig SC, Wu ZJ, Slaughter MS, Giridharan GA*. Sensorless physiologic control, suction prevention, and flow balancing algorithm for rotary biventricular assist devices. IEEE Transactions on Control Systems Technology, 27(2):717-729, 2019.
[41] Koh VCA, Pauls JP, Wu EL, et al., A centralized multi-objective model predictive control for a biventricular assist device: an in vitro evaluation. Biomedical Signal Processing and Control 2020, 59, 101914.
[42] Wang Y, Peng J, Rodefeld MD, Luan Y, Giridharan GA*. A sensorless physiologic control strategy for continuous flow cavopulmonary circulatory support devices. Biomedical Signal Processing and Control, 62(3): 102130, 2020.
[43] Wang Y, Peng J, Qin K, Rodefeld MD, Luan Y, Giridharan GA*. Control strategy to enhance pulmonary vascular pulsatility for implantable cavopulmonary assist device: A simulation study. Biomedical Signal Processing and Control, 70:103008, 2021.
[44] Wang Y, Koenig SC, Slaughter MS, Giridharan GA*. Rotary blood pump control strategy for preventing suction. ASAIO Journal, 61(1):21-30, 2015.
[45] G.A. Giridharan and M. Skliar, “Physiological control of blood pumps using intrinsic pump parameters: A computer simulation study,” Artif. Organs, vol. 30, no. 4, pp. 301-307, Apr. 2006.
[46] S. D. Gregory et al., “In vivo evaluation of active and passive physiological control systems for rotary left and right ventricular assist devices,” Artif. Organs, vol. 40, no. 9, pp. 894-903, Sep. 2016.
[47] M. H. Meki, Y. Wang, P. Sethu, M. Ghazal, A. El-Baz, and G. A. Giridharan, “A Sensorless Rotational Speed-based Control System for Continuous Flow Left Ventricular Assist Devices,” IEEE Trans Biomed Eng., to be published. DOI: 10.1109/TBME.2019.2928826.
[48] Liang, L.; Qin, K.; El-Baz, A. S.; Roussel, T. J.; Sethu, P.; Giridharan, G. A.; Wang, Y. A Flow Sensor-Based Suction-Index Control Strategy for Rotary Left Ventricular Assist Devices. Sensors 2021, 21 (20), 6890. https://doi.org/10.3390/s21206890.
[49] Palagani Y, Sorkin E, Bonde R, Bahel P, Bonde P. Resonantly coupled high-efficiency sensors for assessment of ventricular chamber size for autonomous control of left ventricular assist device. ASAIO Journal, 69 (1): 50-58, 2023.
[50] Choi, S.; Boston, J.R.; Thomas, D.; Antaki, J.F. Modeling and identification of an axial flow blood pump. In Proceedings of the 1997 American Control Conference (ACC), Albuquerque, NM, USA, 6 June 1997; pp. 3714-3715.
[51] Pillay, P.; Krishnan, R. Modeling, simulation and analysis of permanent-magnet motor drives, part II: The brushless DC motor drive. IEEE Trans. Ind. Appl. 1989, 25, 265-273.
[52] Soucy KG, Koenig SC, Sobieksi MA, Slaughter MS, Giridharan GA: Fault detection in rotary blood pumps using motor speed response. ASAIO J 59:410-419, 2013.
[53] Petrou, A.; Kuster, D.; Lee, J.; Meboldt, M.; Daners, M.S. Comparison of flow estimators for rotary blood pumps: An in vitro and in vivo study. Ann. Biomed. Eng. 2018, 46, 2123-2134.
[54] Peng, J., et al. A New Physiological Control Strategy based on the CardioMEMS Pulmonary Artery Pressure Sensor for Rotary Blood Pumps. in 2020 IEEE 16th International Conference on Control & Automation (ICCA). 2020. IEEE.
[55] Theochari, C.A., et al., Heart transplantation versus left ventricular assist devices as destination therapy or bridge to transplantation for 1-year mortality: a systematic review and meta-analysis. Annals of cardiothoracic surgery, 2018. 7(1): p. 3.
[56] Goldstein, D.J., et al., Continuous-flow devices and percutaneous site infections: clinical outcomes. The Journal of heart and lung transplantation, 2012. 31(11): p. 1151-1157.
[57] Timms, D., A review of clinical ventricular assist devices. Medical engineering & physics, 2011. 33(9): p. 1041-1047.
[58] Doost, S.N., L. Zhong, and Y.S. Morsi, Ventricular assist devices: current state and challenges. Journal of Medical Devices, 2017. 11(4): p. 040801.
[59] Slaughter, M.S., et al., Clinical management of continuous-flow left ventricular assist devices in advanced heart failure. The Journal of heart and lung transplantation, 2010. 29 (4): p. S1-S39.
[60] Ochsner, G., et al., In vivo evaluation of physiologic control algorithms for left ventricular assist devices based on left ventricular volume or pressure. Asaio Journal, 2017. 63(5): p. 568-577.
[61] AlOmari, A.-H.H., et al., Developments in control systems for rotary left ventricular assist devices for heart failure patients: a review. Physiological measurement, 2012. 34(1): p. R1.
[62] Petrou, A., et al., A physiological controller for turbodynamic ventricular assist devices based on left ventricular systolic pressure. Artificial organs, 2016. 40(9): p. 842-855.
[63] Wang, Y., et al., Rotary blood pump control strategy for preventing left ventricular suction. ASAIO Journal, 2015. 61(1): p. 21-30.
[64] Gaddum, N.R., et al., Starling-like flow control of a left ventricular assist device: in vitro validation. Artificial organs, 2014. 38(3): p. E46-E56.
[65] Chang, Y., B. Gao, and K. Gu, A model-free adaptive control to a blood pump based on heart rate. Asaio Journal, 2011. 57(4): p. 262-267.
[66] Wu, Y., et al., An advanced physiological controller design for a left ventricular assist device to prevent left ventricular collapse. Artificial organs, 2003. 27(10): p. 926-930.
[67] Wang, Y., et al., Suction prevention and physiologic control of continuous flow left ventricular assist devices using intrinsic pump parameters. ASAIO Journal, 2015. 61(2): p. 170-177.
[68] Giridharan, G.A., et al., Loss of pulsatility with continuous-flow left ventricular assist devices and the significance of the arterial endothelium in von-Willebrand factor production and degradation. Artificial organs, 2023. 47(4): p. 640-648.
[69] Soucy, K.G., et al., Defining pulsatility during continuous-flow ventricular assist device support. The Journal of Heart and Lung Transplantation, 2013. 32(6): p. 581-587.
[70] Meki, M., et al., Effects of pulsatility on arterial endothelial and smooth muscle cells. Cells Tissues Organs, 2023. 212(3): p. 272-284.
[71] Ambardekar, A.V., et al., Coronary artery remodeling and fibrosis with continuous-flow left ventricular assist device support. Circulation: Heart Failure, 2018. 11(5): p. e004491.
[72] Joly, J.M., et al., High right atrial pressure and low pulse pressure predict gastrointestinal bleeding in patients with left ventricular assist device. Journal of cardiac failure, 2018. 24(8): p. 487-493.
[73] Lescroart, M., et al., Pulsatility in ventricular assistance devices: a translational review focused on applied haemodynamics. Archives of cardiovascular diseases, 2020. 113(6-7): p. 461-472.
[74] Palagani, Y., et al., Resonantly coupled high-efficiency sensors for assessment of ventricular chamber size for autonomous control of left ventricular assist device. ASAIO Journal, 2023. 69(1): p. 50-58.
[75] Liang, L., et al., A suction index based control system for rotary blood pumps. Biomedical Signal Processing and Control, 2020. 62: p. 102057.
[76] Meki, M., et al., A sensorless rotational speed-based control system for continuous flow left ventricular assist devices. IEEE Transactions on Biomedical Engineering, 2019. 67(4): p. 1050-1060.
[77] Tan, Z., et al., A sensorless, physiologic feedback control strategy to increase vascular pulsatility for rotary blood pumps. Biomedical signal processing and control, 2023. 83: p. 104640.
[78] Wang, Y., et al., Control strategy to enhance pulmonary vascular pulsatility for implantable cavopulmonary assist devices: A simulation study. Biomedical Signal Processing and Control, 2021. 70: p. 103008.
[79] Wang, Y., et al., Sensor-based physiologic control strategy for biventricular support with rotary blood pumps. Asaio Journal, 2018. 64(3): p. 338-350.
[80] Giridharan, G.A. and M. Skliar. Modeling of the human circulatory system with an axial flow ventricular assist device. in Proceedings of the 2001 American Control Conference. (Cat. No. 01CH37148). 2001. IEEE.
[81] Choi, S., et al. Modeling and identification of an axial flow blood pump. in Proceedings of the 1997 American Control Conference (Cat. No. 97CH36041). 1997. IEEE.
[82] Pillay, P. and R. Krishnan, Modeling, simulation, and analysis of permanent-magnet motor drives. II. The brushless DC motor drive. IEEE transactions on Industry applications, 1989. 25(2): p. 274-279.
[83] Petrou, A., et al., Comparison of flow estimators for rotary blood pumps: An in vitro and in vivo study. Annals of biomedical engineering, 2018. 46: p. 2123-2134.
[84] Giridharan, G.A., et al., Modeling and control of a brushless DC axial flow ventricular assist device. ASAIO journal, 2002. 48(3): p. 272-289.
[85] Meki, M., et al. A sensorless non-linear control algorithm for continuous flow right ventricular assist devices. in 2018 IEEE International Symposium on Signal Processing and Information Technology (ISSPIT). 2018. IEEE.
[86] Wang, Y., et al., Sensorless physiologic control, suction prevention, and flow balancing algorithm for rotary biventricular assist devices. IEEE Transactions on Control Systems Technology, 2017. 27(2): p. 717-729.
[87] Ising, M.S., et al., Feasibility of pump speed modulation for restoring vascular pulsatility with rotary blood pumps. Asaio Journal, 2015. 61(5): p. 526-532.
[88] Sah, J.P., et al., Understanding the role of vascular stretch on modulation of VWF and ANGPT-2 in continuous flow left ventricular assist device (CF-VAD) patients. Lab on a Chip, 2025.
[89] Sun, P., S. Bozkurt, and E. Sorguven, Computational analyses of aortic blood flow under varying speed CF-LVAD support. Computers in biology and medicine, 2020. 127: p. 104058.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. A physiological control system for a blood pump comprising:
a controller configured to:
receive an input signal indicative of ventricular chamber volume, and
generate an output pump control signal based on the input signal.

2. The physiological control system of claim 1, wherein the measured ventricular chamber volume is at least one of end diastolic volume, end systolic volume, mean ventricular volumes, stroke volume, or ventricular volume.

3. The physiological control system of claim 1, wherein the output pump control signal is generated based on the gain-scheduling proportional-integral control equation:
$$I = {K}_{P} ( EDV - {EDV}_{r} ) + {K}_{I} {\int }_{0}^{t} ( EDV - {EDV}_{r} ) dt .$$

4. The physiological control system of claim 1, wherein the output pump control signal is based on weighting ventricular chamber volumes dependent of the part of the cardiac cycle.

5. The physiological control system of claim 1, wherein KP is substantially 0.01 and KI is substantially 0.002 for controlling an axial rotary blood pump.

6. The physiological control system of claim 1, wherein KP is substantially 0.03 and KI is substantially 0.006 for controlling a centrifugal rotary blood pump.

7. The physiological control system of claim 1, wherein the controller calculates setpoints as at least one of a constant setpoint, a repeating continuous or discrete function, or a non-repeating function.

8. The physiological control system of claim 1, wherein the measured chamber volume is based on a signal generated from resonantly coupled sensors.

9. The physiological control system of claim 8, wherein the resonantly coupled sensors comprise apical and outflow sensors.

10. The physiological control system of claim 1, wherein the output pump control signal is a pump speed signal.

11. The physiological control system of claim 1, wherein the controller is configured to detect when end-systolic volumes are above a minimum setpoint.

12. The physiological control system of claim 1, wherein the controller is configured to detect changes in pump power.

13. The physiological control system of claim 1, wherein the controller is configured to periodically switch volume setpoints to generate pulsatility.

14. The physiological control system of claim 1, wherein the controller is configured to periodically set pump speed at a low constant speed for estimating at least one of the ejection fraction, rate of change of volume, ventricular end-systolic and end-diastolic volumes.

15. The physiological control system of claim 1, wherein the controller is configured to use stroke volume as a setpoint that is periodically increased to a larger value.

16. The physiological control system of claim 1, wherein the controller is configured to set pump flow lower than stroke volume.

17. The physiological control system of claim 1, wherein the controller is configured to increase stroke volume as improvement in physiological parameters is detected.

18. A physiological method for controlling a blood pump, the method comprising:
receiving an input signal indicative of ventricular chamber volume; and
generating an output pump control signal based on the input signal.

19. The method of claim 18, wherein the measured ventricular chamber volume is at least one of end diastolic volume, end systolic volume, mean ventricular volumes, stroke volume, or ventricular volume.

20. The method of claim 18, wherein the output pump control signal is generated based on the gain-scheduling proportional-integral control equation:
$$I = {K}_{P} ( EDV - {EDV}_{r} ) + {K}_{I} {\int }_{0}^{t} ( EDV - {EDV}_{r} ) dt .$$

21. The method of claim 18, wherein the output pump control signal is based on weighting ventricular chamber volumes dependent of the part of the cardiac cycle.

22. The method of claim 18, wherein KP is substantially 0.01 and KI is substantially 0.002 for controlling an axial rotary blood pump.

23. The method of claim 18, wherein KP is substantially 0.03 and KI is substantially 0.006 for controlling a centrifugal rotary blood pump.

24. The method of claim 18, wherein the controller calculates setpoints as at least one of a constant setpoint, a repeating continuous or discrete function, or a non-repeating function.

25. The method of claim 18, wherein the measured chamber volume is based on a signal generated from resonantly coupled sensors.

26. The method of claim 25, wherein the resonantly coupled sensors comprise apical and outflow sensors.

27. The method of claim 18, wherein the output pump control signal is a pump speed signal.

28. The method of claim 18 further comprising:
detecting when end-systolic volumes are above a minimum setpoint.

29. The method of claim 18 further comprising:
detecting changes in pump power.

30. The method of claim 18 further comprising:
periodically switching volume setpoints to generate pulsatility.

31. The method of claim 18 further comprising:
periodically setting pump speed at a low constant speed for estimating at least one of the ejection fraction, rate of change of volume, ventricular end-systolic and end-diastolic volumes.

32. The method of claim 18 further comprising:
utilizing stroke volume as a setpoint that is periodically increased to a larger value.

33. The method of claim 18 further comprising:
setting pump flow lower than stroke volume.

34. The method of claim 18 further comprising:
increasing stroke volume as improvement in physiological parameters is detected.
